SEQUENCE LISTING (9 sequences)

SEQ ID NO: 1 - DNA, 3036 bp, Artificial Sequence
  Synthetic
atgggaccaatgggcccaagaggtcctcctggaccagccggtgcaccaggtcctcagggttttcaaggtaacccaggtgagcctggtgagccaggtgtttctggtcctatgggtcctcgtggccctcctggtcctccaggcaagcctggtgatgatggtgaagctggaaagccaggtaagtctggtgaaagaggcccaccaggtccacaaggtgctagaggatttccaggtactcctggtttgcctggtgttaagggtcatagaggttacccaggtttggatggtgctaaaggtgaagccggtgctcccggtgtcaaaggtgaatctggttctcctggtgaaaacggttctcccggacctatgggacctagaggtttacctggtgagagaggtagaactggtccagctggtgctgctggtgccagaggaaatgatggacaaccaggacctgctggtcctcctggtcctgttggtccagctggcggtccaggatttcctggtgctccaggtgcaaaaggcgaagctggacctactggtgctaggggtcctgaaggtgctcaaggacccagaggcgaacctggaacacccggatctccaggtcctgccggcgctgctggtaatcctggtactgatggaataccaggtgctaagggttctgctggcgctcctggaatagctggtgcccctggttttccaggacctaggggtcctccaggacctcaaggtgctactggaccattaggtcctaaaggtcagactggcgaaccaggtattgctggtttcaagggtgaacagggaccaaaaggtgagcccggtccagccggaccacagggtgccccaggaccagctggcgaagagggtaaacgtggtgcaaggggtgaacccggtggtgccggtcctgcaggcccacctggtgaacgtggcgccccaggtaatagaggattccctggacaagatggtttggccggtcctaagggcgcaccaggtgaaaggggtccatctggtttagctggtcccaaaggtgcaaatggtgatccaggtagaccaggcgagccaggtttgccaggtgctcgtggattaactggtagacctggtgacgctggaccacaaggtaaagttggtccatcaggtgcacccggtgaagatggtagacccggtcctccaggtccacagggcgcaagaggtcaacctggtgttatgggattccccggacctaagggtgctaatggcgagcctggaaaagccggtgaaaaaggattgccaggcgctccaggacttagaggacttcctggtaaggacggtgaaacaggtgctgccggtccacctggacctgctggacccgctggtgaaaggggcgaacaaggtgcaccaggaccttccggatttcaaggtctgcccggaccacctggtcctccaggtgaaggtggtaaaccaggtgatcaaggtgtccctggtgaggctggcgcacctggtctagttggaccaaggggcgagcgtggatttcccggcgaaagaggttctccaggttctcaaggattgcaaggtgccaggggattgcccggaactcccggtacagacggccctaaaggtgcagcaggccccgcaggacctcctggcgcacaaggccctccaggattgcagggtatgccaggcgagagaggtgcagctggtattgcaggacccaagggtgatcgtggtgatgttggagaaaaaggcccagaaggcgctcccggtaaggatggtggtagaggtttgactggtccaattggcccacctggtccagccggcgctaatggtgaaaaaggtgaagttggcccaccaggacctgccggtacagctggtgctagaggtgccccaggtgaaagaggtgaaacaggacctccaggtccagccggttttgctggtccaccaggtgctgatggtcagcctggtgccaagggcgagcagggtgaagcaggtcaaaagggcgacgcaggcgcccctggacctcagggtcctagcggagccccaggtcctcaaggaccaactggtgttactggacctaaaggcgccagaggtgcacagggtccaccaggcgctactggtttcccaggtgccgctggtagagttggacctcctggttctaatggtaatcccggacctcctggaccacctggtccatctggtaaagatggacctaaaggtgctcgtggtgattctggtccacctggcagagctggtgaccctggattgcaaggccctgccggaccaccaggcgaaaagggcgaacctggtgatgacggtccttctggtccagatggccctccaggaccacaaggattggctggtcaaagaggtatcgttggtttgccaggacaaagaggtgagagaggattcccaggtttaccaggaccatcaggcgagcccggtaaacaaggcgcacccggcgctagcggtgataggggccctcctggacctgtgggaccaccaggattgacaggacctgcaggcgaaccaggcagagaaggtagtccaggtgccgacggtccaccaggtagagatggcgctgctggtgtaaaaggtgataggggtgaaactggtgcagttggagccccaggcgctcccggacctccaggttctcctggacctgcaggcccaattggtaaacaaggtgatagaggtgaggccggtgcacaaggaccaatgggtcccgccggtcctgccggtgcaagaggtatgcctggtccacagggtccccgtggtgacaaaggtgagacaggtgaagctggcgaaagaggattgaagggacacagaggttttaccggtctgcaaggattacccggtccacctggtccaagtggtgatcaaggtgcttctggtcctgctggtccctctggaccaagaggcccacctggacctgtcggtccttctggaaaagatggtgctaacggtattccaggtccaattggtccaccaggacctagaggtagatctggtgaaaccggacctgctggacctccaggtaaccctggtcctcctggtccaccaggacctcca

SEQ ID NO: 2 - protein, 1012 aa, Artificial Sequence
  Synthetic
MGPMGPRGPPGPAGAPGPQGFQGNPGEPGEPGVSGPMGPRGPPGPPGKPGDDGEAGKPGKSGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGAAGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGAGPAGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGSQGLQGARGLPGTPGTDGPKGAAGPAGPPGAQGPPGLQGMPGERGAAGIAGPKGDRGDVGEKGPEGAPGKDGGRGLTGPIGPPGPAGANGEKGEVGPPGPAGTAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGDPGLQGPAGPPGEKGEPGDDGPSGPDGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPIGKQGDRGEAGAQGPMGPAGPAGARGMPGPQGPRGDKGETGEAGERGLKGHRGFTGLQGLPGPPGPSGDQGASGPAGPSGPRGPPGPVGPSGKDGANGIPGPIGPPGPRGRSGETGPAGPPGNPGPPGPPGPP

SEQ ID NO: 3 - DNA, 1500 bp, Artificial Sequence
  Synthetic
atgggaccaatgggcccaagaggtcctcctggaccagccggtgcaccaggtcctcagggttttcaaggtaacccaggtgagcctggtgagccaggtgtttctggtcctatgggtcctcgtggccctcctggtcctccaggcaagcctggtgatgatggtgaagctggaaagccaggtaagtctggtgaaagaggcccaccaggtccacaaggtgctagaggatttccaggtactcctggtttgcctggtgttaagggtcatagaggttacccaggtttggatggtgctaaaggtgaagccggtgctcccggtgtcaaaggtgaatctggttctcctggtgaaaacggttctcccggacctatgggacctagaggtttacctggtgagagaggtagaactggtccagctggtgctgctggtgccagaggaaatgatggacaaccaggacctgctggtcctcctggtcctgttggtccagctggcggtccaggatttcctggtgctccaggtgcaaaaggcgaagctggacctactggtgctaggggtcctgaaggtgctcaaggacccagaggcgaacctggaacacccggatctccaggtcctgccggcgctgctggtaatcctggtactgatggaataccaggtgctaagggttctgctggcgctcctggaatagctggtgcccctggttttccaggacctaggggtcctccaggacctcaaggtgctactggaccattaggtcctaaaggtcagactggcgaaccaggtattgctggtttcaagggtgaacagggaccaaaaggtgagcccggtccagccggaccacagggtgccccaggaccagctggcgaagagggtaaacgtggtgcaaggggtgaacccggtggtgccggtcctgcaggcccacctggtgaacgtggcgccccaggtaatagaggattccctggacaagatggtttggccggtcctaagggcgcaccaggtgaaaggggtccatctggtttagctggtcccaaaggtgcaaatggtgatccaggtagaccaggcgagccaggtttgccaggtgctcgtggattaactggtagacctggtgacgctggaccacaaggtaaagttggtccatcaggtgcacccggtgaagatggtagacccggtcctccaggtccacagggcgcaagaggtcaacctggtgttatgggattccccggacctaagggtgctaatggcgagcctggaaaagccggtgaaaaaggattgccaggcgctccaggacttagaggacttcctggtaaggacggtgaaacaggtgctgccggtccacctggacctgctggacccgctggtgaaaggggcgaacaaggtgcaccaggaccttccggatttcaaggtctgcccggaccacctggtcctccaggtgaaggtggtaaaccaggtgatcaaggtgtccctggtgaggctggcgcacctggtctagttggaccaaggggcgagcgtgga

SEQ ID NO: 4 - protein, 500 aa, Artificial Sequence
  Synthetic
MGPMGPRGPPGPAGAPGPQGFQGNPGEPGEPGVSGPMGPRGPPGPPGKPGDDGEAGKPGKSGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGAAGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGAGPAGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERG

SEQ ID NO: 5 - DNA, 765 bp, Artificial Sequence
  Synthetic
atgaggcaggtttggttctcttggattgtgggattgttcctatgttttttcaacgtgtcttctgctcatcatcaccaccatcacatggttctgtccaaatcctgtgtttcacacttccgaaatgtaggttctctaaacagtagagatgttaatcttaaagacgatttcagctatgccaatattgatgatccttacaataagccatttgtgcttaataacctgattaaccctactaagtgtcaagaaatcatgcaatttgcaaatggcaagttgtttgattcacaggttttgtctgggactgacaagaacattagaaactcgcaacaaatgtggattagcaagaacaatcctatggtaaagccaatctttgagaatatttgcagacagttcaatgttccctttgacaatgctgaagatttgcaagtcgttcgttatcttccaaaccagtactataacgaacaccatgactcgtgttgtgactcttcaaaacaatgcagtgagttcattgagagaggtggtcaaaggattttgacagtcttgatctacctaaacaatgagttcagtgatggacacacttactttccgaacttaaaccagaaattcaagcccaaaactggagatgcattagtcttttatccattggccaataactctaacaaatgccatccatactccttacatgctggtatgcctgtaacatctggagaaaagtggattgctaatttgtggtttagagaacgtaaattctcccatgatgaattgtaa

SEQ ID NO: 6 - protein, 254 aa, Artificial Sequence
  Synthetic
MRQVWFSWIVGLFLCFFNVSSAHHHHHHMVLSKSCVSHFRNVGSLNSRDVNLKDDFSYANIDDPYNKPFVLNNLINPTKCQEIMQFANGKLFDSQVLSGTDKNIRNSQQMWISKNNPMVKPIFENICRQFNVPFDNAEDLQVVRYLPNQYYNEHHDSCCDSSKQCSEFIERGGQRILTVLIYLNNEFSDGHTYFPNLNQKFKPKTGDALVFYPLANNSNKCHPYSLHAGMPVTSGEKWIANLWFRERKFSHDEL

SEQ ID NO: 7 - DNA, 7311 bp, Artificial Sequence
  Synthetic
ggaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgataggctcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcagggcgcgccgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctatttaaatgggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgaggcaggtttggttctcttggattgtgggattgttcctatgttttttcaacgtgtcttctgctcctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctatgggaccaatgggcccaagaggtcctcctggaccagccggtgcaccaggtcctcagggttttcaaggtaacccaggtgagcctggtgagccaggtgtttctggtcctatgggtcctcgtggccctcctggtcctccaggcaagcctggtgatgatggtgaagctggaaagccaggtaagtctggtgaaagaggcccaccaggtccacaaggtgctagaggatttccaggtactcctggtttgcctggtgttaagggtcatagaggttacccaggtttggatggtgctaaaggtgaagccggtgctcccggtgtcaaaggtgaatctggttctcctggtgaaaacggttctcccggacctatgggacctagaggtttacctggtgagagaggtagaactggtccagctggtgctgctggtgccagaggaaatgatggacaaccaggacctgctggtcctcctggtcctgttggtccagctggcggtccaggatttcctggtgctccaggtgcaaaaggcgaagctggacctactggtgctaggggtcctgaaggtgctcaaggacccagaggcgaacctggaacacccggatctccaggtcctgccggcgctgctggtaatcctggtactgatggaataccaggtgctaagggttctgctggcgctcctggaatagctggtgcccctggttttccaggacctaggggtcctccaggacctcaaggtgctactggaccattaggtcctaaaggtcagactggcgaaccaggtattgctggtttcaagggtgaacagggaccaaaaggtgagcccggtccagccggaccacagggtgccccaggaccagctggcgaagagggtaaacgtggtgcaaggggtgaacccggtggtgccggtcctgcaggcccacctggtgaacgtggcgccccaggtaatagaggattccctggacaagatggtttggccggtcctaagggcgcaccaggtgaaaggggtccatctggtttagctggtcccaaaggtgcaaatggtgatccaggtagaccaggcgagccaggtttgccaggtgctcgtggattaactggtagacctggtgacgctggaccacaaggtaaagttggtccatcaggtgcacccggtgaagatggtagacccggtcctccaggtccacagggcgcaagaggtcaacctggtgttatgggattccccggacctaagggtgctaatggcgagcctggaaaagccggtgaaaaaggattgccaggcgctccaggacttagaggacttcctggtaaggacggtgaaacaggtgctgccggtccacctggacctgctggacccgctggtgaaaggggcgaacaaggtgcaccaggaccttccggatttcaaggtctgcccggaccacctggtcctccaggtgaaggtggtaaaccaggtgatcaaggtgtccctggtgaggctggcgcacctggtctagttggaccaaggggcgagcgtggatttcccggcgaaagaggttctccaggttctcaaggattgcaaggtgccaggggattgcccggaactcccggtacagacggccctaaaggtgcagcaggccccgcaggacctcctggcgcacaaggccctccaggattgcagggtatgccaggcgagagaggtgcagctggtattgcaggacccaagggtgatcgtggtgatgttggagaaaaaggcccagaaggcgctcccggtaaggatggtggtagaggtttgactggtccaattggcccacctggtccagccggcgctaatggtgaaaaaggtgaagttggcccaccaggacctgccggtacagctggtgctagaggtgccccaggtgaaagaggtgaaacaggacctccaggtccagccggttttgctggtccaccaggtgctgatggtcagcctggtgccaagggcgagcagggtgaagcaggtcaaaagggcgacgcaggcgcccctggacctcagggtcctagcggagccccaggtcctcaaggaccaactggtgttactggacctaaaggcgccagaggtgcacagggtccaccaggcgctactggtttcccaggtgccgctggtagagttggacctcctggttctaatggtaatcccggacctcctggaccacctggtccatctggtaaagatggacctaaaggtgctcgtggtgattctggtccacctggcagagctggtgaccctggattgcaaggccctgccggaccaccaggcgaaaagggcgaacctggtgatgacggtccttctggtccagatggccctccaggaccacaaggattggctggtcaaagaggtatcgttggtttgccaggacaaagaggtgagagaggattcccaggtttaccaggaccatcaggcgagcccggtaaacaaggcgcacccggcgctagcggtgataggggccctcctggacctgtgggaccaccaggattgacaggacctgcaggcgaaccaggcagagaaggtagtccaggtgccgacggtccaccaggtagagatggcgctgctggtgtaaaaggtgataggggtgaaactggtgcagttggagccccaggcgctcccggacctccaggttctcctggacctgcaggcccaattggtaaacaaggtgatagaggtgaggccggtgcacaaggaccaatgggtcccgccggtcctgccggtgcaagaggtatgcctggtccacagggtccccgtggtgacaaaggtgagacaggtgaagctggcgaaagaggattgaagggacacagaggttttaccggtctgcaaggattacccggtccacctggtccaagtggtgatcaaggtgcttctggtcctgctggtccctctggaccaagaggcccacctggacctgtcggtccttctggaaaagatggtgctaacggtattccaggtccaattggtccaccaggacctagaggtagatctggtgaaaccggacctgctggacctccaggtaaccctggtcctcctggtccaccaggacctccataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctcc

SEQ ID NO: 8 - DNA, 5154 bp, Artificial Sequence
  Synthetic
ggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgataggctcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggacccaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgaggcaggtttggttctcttggattgtgggattgttcctatgttttttcaacgtgtcttctgctcctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagagaggccgaagctatgggaccaatgggcccaagaggtcctcctggaccagccggtgcaccaggtcctcagggttttcaaggtaacccaggtgagcctggtgagccaggtgtttctggtcctatgggtcctcgtggccctcctggtcctccaggcaagcctggtgatgatggtgaagctggaaagccaggtaagtctggtgaaagaggcccaccaggtccacaaggtgctagaggatttccaggtactcctggtttgcctggtgttaagggtcatagaggttacccaggtttggatggtgctaaaggtgaagccggtgctcccggtgtcaaaggtgaatctggttctcctggtgaaaacggttctcccggacctatgggacctagaggtttacctggtgagagaggtagaactggtccagctggtgctgctggtgccagaggaaatgatggacaaccaggacctgctggtcctcctggtcctgttggtccagctggcggtccaggatttcctggtgctccaggtgcaaaaggcgaagctggacctactggtgctaggggtcctgaaggtgctcaaggacccagaggcgaacctggaacacccggatctccaggtcctgccggcgctgctggtaatcctggtactgatggaataccaggtgctaagggttctgctggcgctcctggaatagctggtgcccctggttttccaggacctaggggtcctccaggacctcaaggtgctactggaccattaggtcctaaaggtcagactggcgaaccaggtattgctggtttcaagggtgaacagggaccaaaaggtgagcccggtccagccggaccacagggtgccccaggaccagctggcgaagagggtaaacgtggtgcaaggggtgaacccggtggtgccggtcctgcaggcccacctggtgaacgtggcgccccaggtaatagaggattccctggacaagatggtttggccggtcctaagggcgcaccaggtgaaaggggtccatctggtttagctggtcccaaaggtgcaaatggtgatccaggtagaccaggcgagccaggtttgccaggtgctcgtggattaactggtagacctggtgacgctggaccacaaggtaaagttggtccatcaggtgcacccggtgaagatggtagacccggtcctccaggtccacagggcgcaagaggtcaacctggtgttatgggattccccggacctaagggtgctaatggcgagcctggaaaagccggtgaaaaaggattgccaggcgctccaggacttagaggacttcctggtaaggacggtgaaacaggtgctgccggtccacctggacctgctggacccgctggtgaaaggggcgaacaaggtgcaccaggaccttccggatttcaaggtctgcccggaccacctggtcctccaggtgaaggtggtaaaccaggtgatcaaggtgtccctggtgaggctggcgcacctggtctagttggaccaaggggcgagcgtggataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgctccgga

SEQ ID NO: 9 - DNA, 4348 bp, Artificial Sequence
  Synthetic
atttaaatagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgaggcaggtttggttctcttggattgtgggattgttcctatgttttttcaacgtgtcttctgctcatcatcaccaccatcacatggttctgtccaaatcctgtgtttcacacttccgaaatgtaggttctctaaacagtagagatgttaatcttaaagacgatttcagctatgccaatattgatgatccttacaataagccatttgtgcttaataacctgattaaccctactaagtgtcaagaaatcatgcaatttgcaaatggcaagttgtttgattcacaggttttgtctgggactgacaagaacattagaaactcgcaacaaatgtggattagcaagaacaatcctatggtaaagccaatctttgagaatatttgcagacagttcaatgttccctttgacaatgctgaagatttgcaagtcgttcgttatcttccaaaccagtactataacgaacaccatgactcgtgttgtgactcttcaaaacaatgcagtgagttcattgagagaggtggtcaaaggattttgacagtcttgatctacctaaacaatgagttcagtgatggacacacttactttccgaacttaaaccagaaattcaagcccaaaactggagatgcattagtcttttatccattggccaataactctaacaaatgccatccatactccttacatgctggtatgcctgtaacatctggagaaaagtggattgctaatttgtggtttagagaacgtaaattctcccatgatgaattgtaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgagtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2024/0182546 A1
(43) Pub. Date: Jun. 6, 2024

(54) RECOMBINANT TYPE II COLLAGEN FOR THERAPEUTIC USE

(71) Applicant: Gelita AG, Eberbach (DE)

(72) Inventors: Stephan Hausmanns, Heidelberg (DE); Hans-Ulrich Frech, Weinheim (DE); Steffen Oesser, GlÜcksburg (DE); Martin Hahn, Gronau (DE)

(21) Appl. No.: 18/283,653

(22) PCT Filed: Mar. 23, 2022
(86) PCT No.: PCT/EP2022/057624

§ 371 Date: Sep. 22, 2023

(30) Foreign Application Priority Data
Mar. 23, 2021 (DE) 10 2021 202 830.6

(51) Int. Cl. C07K 14/78; A61K 9/00; A61K 38/39; A61P 19/02; C12N 15/81

(52) U.S. Cl. CPC C07K 14/78; A61K 9/0053; A61K 38/39; A61P 19/02; C12N 15/815

(57) ABSTRACT

The present invention relates to recombinant, in particular non-denatured, type II collagen for use in a therapeutic method for oral therapy of cartilage disease of a human or animal patient.

The following invention relates to recombinant type II collagen for use in a therapeutic method for oral therapy of cartilage diseases of a human or animal body.

Collagen is an extracellular structural protein found in animals, for example mammals, birds and fish. It is usually found there in connective tissue, especially as a component of the extracellular matrix. Tendons, ligaments, cartilage and bones are particularly rich in collagen. In contrast, collagens are not naturally found in plants and unicellular organisms.

Collagens occur in various, structurally and functionally different types and differ, among other things, in terms of their structure, function and origin. The polypeptide chains that build up collagen are synthesised individually in the cell at the ribosomes of the endoplasmic reticulum in the form of larger precursor molecules and have extensive repetitive (Gly-X—Y)n sequences, wherein X and Y can be any amino acid, but are usually proline and 4-hydroxyproline.

These precursor polypeptide chains are hydroxylated post-translationally at proline and lysine residues of the polypeptide chain in the endoplasmic reticulum to form hydroxyproline and hydroxylysine residues. Hydroxylation serves to stabilise neighbouring collagen polypeptide chains of the right-handed triple helix formed in the cell from three of each precursor polypeptide chain (procollagen).

The procollagen formed in this way is glycosylated intracellularly, secreted by the cell in the glycosylated triple-helical form (tropocollagen) and then mature collagen is formed by peptidase-mediated cleavage of the terminal residues. In a process of fibrillogenesis, this collagen assembles into collagen fibrils, which are then covalently cross-linked to form collagen fibres.

Collagen is also frequently used in denatured form, sometimes referred to as gelatine, or in the form of its hydrolysates.

If gelatine or collagen is subjected to hydrolytic processes, in particular enzymatic hydrolysis, to obtain collagen peptides, collagen hydrolysates with a wide variety of compositions and application profiles can be produced, depending on the type of collagen used, its origin and the enzymatic conditions. However, these collagen hydrolysates represent a mixture of peptides whose molecular weights are distributed over certain size ranges. The use of such collagen hydrolysates, for example as food supplements or as cosmetic aids, has been known for some time, inter alia for the prevention and/or treatment of ailments related to the bones, joints or connective tissue.

For example, WO 2012/065782 describes collagen hydrolysates obtained from pork rind gelatine, which serve to stimulate the biosynthesis of extracellular matrix proteins by skin cells and are particularly suitable for cosmetic purposes. WO 2012/117012 discloses enzymatically hydrolysed collagen from bovine split with an average molecular weight of 1500 to 8000 Da, which can be used together with a prebiotic for the prevention and/or treatment of osteoporosis.

Although for many applications and consumer groups the use of collagen or collagen hydrolysates obtained from animal materials has advantages, for certain consumer groups and application profiles the use of such collagen hydrolysates may be less desirable. For example, certain consumer groups are fundamentally critical of or opposed to raw materials obtained from animal materials, either because they fear contamination with microorganisms or agents that are hazardous to health, for example process excipients, or undesirable immune reactions, or because of religious or ethical motives. Furthermore, the production processes used to obtain collagen hydrolysates obtained from animal materials often comprise complex and expensive digestion, purification and further processing steps.

Against this background, it is not surprising that methods have been developed to produce gelatine, collagen, collagen hydrolysates and individual collagen peptides by biotechnological means using recombinant gene technology.

For example, WO 2006/052451 A2 discloses the production of recombinant collagen type III in Pichia pastoris strains that also express human prolyl hydroxylases. WO 2005/012356 A2 discloses the production of gelatin from human collagen type I and individual 50 kDa, 65 kDa and 100 kDa collagen peptide species, each in fully hydroxylated, partially hydroxylated and non-hydroxylated forms. Olsen et al (Protein Expression and Purification, 2005, 40, pg. 346-357) discloses the recombinant production of an 8.5 kDa collagen peptide species from the al chain of human collagen in Pichia pastoris. WO 01/34646 A2 also discloses the production of individual recombinant gelatin species, each with a defined molecular weight of up to 350 kDa resulting from the recombinant production pathway, which may be present in non-hydroxylated, partially hydroxylated or fully hydroxylated form.

Type II collagen is a collagen found specifically in cartilage tissue and is usually present there in the form of a homotrimer of al chains. The production of recombinant type II collagen peptides, their hydroxylation by means of a prolyl-4-hydroxylase and their hydrogenation and formation into procollagen and the accompanying triple helix formation is described, for example, in U.S. Pat. No. 5,593,859.

U.S. Pat. No. 5,399,347 describes the oral administration of water-soluble highly purified type II collagen from natural sources for the treatment of arthritis. However, the production of this collagen is extremely difficult, costly and also susceptible to contamination, especially by microbes.

From U.S. Pat. Nos. 5,750,144, 5,645,851, 5,529,786 and 5,637,321 it is known to provide animal tissue-containing compositions containing type II collagen and to apply them orally to treat rheumatoid arthritis. In particular, these documents disclose the use of cartilage tissue-containing material obtained from natural animal sources to obtain therefrom a type II collagen-containing composition by means of various chemical-physical method steps. The type II collagen-containing compositions thus obtained are characterised by the presence, on the one hand, of non-denatured type II collagen and, on the other hand, of a number of minor components originated from the starting material and the isolation process, in particular proteins, antigens and salts. Although the provision of these compositions requires less complicated method steps than the production of highly purified type II collagens from natural sources, the produced preparations vary from starting material to starting material due to their origin from natural sources. In addition, it is necessary to ensure throughout the production and processing process that the type II collagen contained in the composition is non-denatured and contamination with pathogens is avoided.

U.S. Pat. No. 7,083,820 and EP 1 435 906 disclose the use of methods for obtaining animal tissue-containing compositions containing non-denatured type II collagen, wherein specific method steps are used to eliminate microbial contaminants while at the same time maintaining the original, non-denatured form of the type II collagen.

Also, in view of the increased demands in large parts of the population with regard to health, mobility and fitness, each also in advanced age, there is still a great need for foodstuffs, food supplements and pharmaceutical compositions for improving and/or maintaining cartilage health and for the therapy of cartilage diseases.

The present invention is therefore based on the technical problem of providing type II collagen which overcomes the aforementioned disadvantages, in particular which can be produced in a standardised, reliable and well-defined form, also on a larger industrial and cost-effective scale, and which is suitable for use in a method for oral therapy of cartilage diseases of the human or animal body and/or for maintaining cartilage health, due to its biological effectiveness.

The present invention solves its underlying technical problem by providing the teachings of the independent claims, in particular also the teachings of the preferred embodiments in the description and dependent claims.

The present invention relates to a recombinant type II collagen, in particular a non-denatured recombinant type II collagen, for use in a therapeutic method for oral therapy of cartilage diseases of a human or animal patient.

The present invention is based on the surprising teaching that recombinantly produced type II collagen, in particular also in isolated form, is capable of treating cartilage diseases after oral application. Although the materials and method steps necessary in the prior art for the production of therapeutically effective type II collagen compositions, in particular the use of natural animal cartilage tissues and the use of certain method steps for obtaining the nativity of the collagen present in this starting tissue, are not used or are not carried out and, moreover, the minor components present in the starting material are not present in the recombinantly produced type II collagen according to the invention, a biological effectiveness, in particular a surprisingly high biological effectiveness, of the recombinant type II collagen produced according to the present method was shown. Surprisingly, the recombinant type II collagen provided according to the present invention, in particular recombinant type II collagen peptides, is capable of exhibiting a biological effectiveness, in particular one at least equal to that exhibited by type II collagen obtained from natural sources, and in particular even an improved biological effectiveness is provided. In a preferred embodiment, the present invention enables cartilage diseases in human or animal patients to be treated at very low dosages, i.e. low concentrations of type II collagen, in particular type II collagen peptide, due to the high biological effectiveness of the recombinant type II collagen, in particular type II collagen peptide.

In a particularly preferred embodiment, the recombinant type II collagens, in particular type II collagen peptides, provided according to the invention are capable of treating immune-modulated cartilage diseases, in particular an autoimmune disease, in particular polychondritis or rheumatoid arthritis.

In a particularly preferred embodiment, an immune-modulated cartilage disease is a disease characterised by immune intolerance.

The present invention also relates to recombinant type II collagen, in particular recombinant type II collagen peptides, for the treatment of cartilage diseases, wherein these may be inflammatory and/or degenerative cartilage diseases, in particular rheumatoid arthritis or arthrosis.

The present invention also relates to recombinant type II collagen, in particular recombinant type II collagen peptides, for inducing oral tolerance, in particular for inducing oral tolerance to endogenously present collagen, in particular endogenously present type II collagen, in particular endogenous type II collagen present in cartilage tissue.

In particular, the present invention relates to recombinant type II collagen, in particular recombinant type II collagen peptides and compositions comprising recombinant type II collagen, in particular recombinant type II collagen peptides for use in a method for therapeutic treatment or therapeutic prevention of immune intolerance to type II collagen, in particular by induction of immune tolerance to type II collagen.

The present invention also relates to recombinant type II collagen, in particular recombinant type II collagen peptides, for use in a method of inducing immune tolerance to type II collagen, in particular a composition the administration of which results in the induction of oral tolerance to type II collagen.

The teaching of the present invention therefore advantageously provides a reproducible, biotechnologically producible recombinant type II collagen for oral therapy of cartilage diseases, which can be produced in a standardised manner, the production of which can be carried out on an industrial scale, and which can be produced contamination-free in high purity and yield without being limited by natural starting materials, and which is characterised in particular by the fact that it can be used in low dosage due to its high biological effectiveness.

The recombinant type II collagens used according to the invention, in particular recombinant type II collagen peptides, are characterised by a biological effectiveness which develops after oral administration in human or animal bodies, in particular an immune-modulating and/or inflammation-modulating biological effectiveness. In a particularly preferred embodiment, this biological effectiveness is present in particular for full-length recombinant type II collagens present in non-denatured, i.e. native, form, but in a preferred embodiment also for collagen peptides of shortened length present in the form of recombinant type II collagen peptides.

Preferably, the type II collagens provided according to the invention, in particular type II collagen peptides, are capable of immune-modulation and/or induction of oral tolerance, in particular they cause an immune response and/or induction of oral tolerance in the treated human or animal body.

The recombinant type II collagens provided according to the invention preferably develop a biological effectiveness suppressing the synthesis of immunoglobulins and/or an anti-inflammatory biological effectiveness. Thus, according to the invention, the reduction of pro-inflammatory and the stimulation of anti-inflammatory cytokines could be established. Without being bound by theory, it appears that the orally applied recombinant type II collagen, in particular recombinant type II collagen peptides, provided according to the invention, survives the gastrointestinal passage completely or largely unharmed and causes in immune-modulating cells, in particular immune suppressor cells, in particular cells of the Peyer's plaque, immune-modulating and/or cytokine-regulating reactions and/or signalling cascades which reduce or completely prevent undesired immune reactions and inflammatory processes in the region of cartilage, in particular joint cartilage.

Thus, it is known that endogenous type II collagen or fragments thereof, which is present in or on damaged or degenerated cartilage tissues, can induce an autoimmune reaction, which leads to inflammation, immunoglobulin formation and degenerative and destructive processes in the cartilage, which contribute to further cartilage destruction and degeneration and ultimately cause the appearance of arthrosis, rheumatoid arthritis and similar diseases. The oral application of recombinant type II collagen, in particular type II collagen peptides, provided according to the invention appears to cause an oral tolerance to endogenously present type II collagen triggering such undesirable reactions, so that a therapy of cartilage diseases is made possible.

The recombinant type II collagens, in particular recombinant type II collagen peptides, provided according to the invention are preferably endowed with the ability to interact with cells of the treated human or animal patient, in particular with cells of Peyer's plaque, and in particular to lead to the stimulation of anti-inflammatory and to the inhibition of pro-inflammatory cytokines as well as inhibition of immunoglobulins.

Preferably, the recombinant type II collagens provided according to the invention, in particular recombinant type II collagen peptides, show an inducing effect on the differentiation of peripheral blood monocytes into immunosuppressive M2 macrophages.

In a further preferred embodiment, the recombinant type II collagens, in particular recombinant type II collagen peptides, provided according to the invention lead to a reduction in the synthesis of inflammatory cytokines, in particular TNFα and IFNY, and/or to an induction of the synthesis of anti-inflammatory cytokines, in particular IL-10.

According to a preferred embodiment of the present invention, the recombinant type II collagens provided according to the invention, in particular recombinant type II collagen peptides, lead to a stimulation/induction of the differentiation of naive CD4⁺ T precursor cells into T suppressor cells. Particularly preferably, the stimulation/induction of the differentiation of naive CD4⁺ T precursor cells into T suppressor cells results in an increased release of anti-inflammatory cytokines, preferably IL-10, IL-4 and/or TGF-ß.

In a preferred embodiment of the present invention, the recombinant type II collagens provided according to the invention, in particular recombinant type II collagen peptides, cause a reduced expression of pro-inflammatory cytokines, preferably of IL-1ß, TNFα and/or IL-6, by chondrocytes, in particular by articular chondrocytes.

In a particularly preferred embodiment of the present invention, the recombinant type II collagen is present in a non-denatured, i.e. native, form.

In a particularly preferred embodiment of the present invention, the recombinant type II collagen is present in triple-helical form.

In a preferred embodiment, the present invention provides a recombinant type II collagen which may be present in the form of a type II collagen peptide, i.e. in single-stranded form, or which may be present in multi-stranded form, for example double- or triple-stranded form, also referred to herein as triple-helical form, in particular in the form of a type II procollagen or mature type II collagen, in particular in the form of a homotrimer of type II-al chains.

Provided that the recombinant type II collagen according to the invention is not present as a single-stranded type II collagen peptide, but is present in, for example, triple-helical form, one or all of the individual collagen peptides constituting the triple-helical form of the recombinant type II collagen may be embodied according to the present invention. In particular, the embodiments disclosed in the present teaching relating to recombinant type II collagen peptides also apply to type II collagens which have one, two or three such single-stranded type II collagen peptides, in particular are composed entirely of these, in particular consists of the recombinant type II collagen peptides according to the invention.

In a particularly preferred embodiment, the recombinant type II collagen may be bovine type II collagen (type IIB collagen).

In another particularly preferred embodiment, the recombinant type II collagen may be present in the form of type II procollagen or mature type II collagen.

In a further particularly preferred embodiment, the recombinant type II collagen may be present in triple-helical form, in particular in the form of a homotrimer of three type II-al chains.

In a particularly preferred embodiment, the recombinant type II collagen is present in non-denatured form, also referred to herein as native form, that is, it has the naturally occurring tertiary and quaternary protein structure.

In a particularly preferred embodiment, the recombinant type II collagen may be present in the form of cross-linked or non-cross-linked fibrils.

In a particularly preferred embodiment, the recombinant type II collagen, in particular type II collagen peptide, may be a full-length collagen peptide, i.e. have the complete amino acid sequence of a naturally occurring collagen peptide of type II.

In another particularly preferred embodiment, the recombinant type II collagen is present in the form of a type II collagen peptide.

In a particularly preferred embodiment, the recombinant type II collagen, in particular type II collagen peptide, may be a collagen peptide of type II with a molecular weight in a range from 5 to 400 kDa, in particular 10 to 390 kDa, in particular 10 to 350 kDa, in particular 10 to 300 kDa, in particular 10 to 110 kDa, in particular 40 to 110 kDa, in particular 40 to 100 kDa, in particular 11 to 105 kDa, in particular 15 to 100 kDa, in particular 20 to 99 kDa, in particular 25 to 95 kDa, in particular 30 to 95 kDa, in particular 35 to 95 kDa. Preferably, the recombinant type II collagen, in particular collagen peptide, has a molecular weight in a range from 40 to 50 kDa, in particular 45 kDa.

In a particularly preferred embodiment, the recombinant type II collagen peptide may be present in triple-helical form.

In a particularly preferred embodiment, the recombinant type II collagen, in particular the recombinant type II collagen peptide, of the present invention is fully or partially hydroxylated, fully or partially glycolyzed, or fully or partially hydroxylated and glycolyzed.

In a particularly preferred embodiment, the recombinant type II collagen, in particular the recombinant type II collagen peptide, according to the present invention is a non-hydroxylated type II collagen, in particular type II collagen peptide.

In a further preferred embodiment, the recombinant type II collagen, in particular the recombinant type II collagen peptide, according to the present invention is a hydroxylated type II collagen, in particular type II collagen peptide.

Preferably, the recombinant type II collagen, in particular the recombinant produced type II collagen peptide, has hydroxylated proline and/or hydroxylated lysine.

Preferably, the recombinant type II collagen, in particular the recombinant type II collagen peptide, is a non-hydroxylated, partially hydroxylated or fully hydroxylated type II collagen, in particular type II collagen peptide.

According to a preferred embodiment of the present invention, the recombinant type II collagen, in particular the recombinant type II collagen peptide, is glycosylated. Preferably, the recombinant type II collagen, in particular the recombinant type II collagen peptide, is glycosylated at at least one hydroxylated lysine. Preferably, each hydroxylated lysine of the recombinant type II collagen, in particular the recombinant type II collagen peptide, is glycosylated.

In a preferred embodiment of the present invention, the recombinant type II collagen, in particular recombinant type II collagen peptide, has no amino acid modification, in particular no hydroxylation. Particularly preferably, the recombinant type II collagen, in particular type II collagen peptide, has no hydroxylated and/or glycosylated amino acids.

Preferably, the type II collagen according to the invention, in particular type II collagen peptide, has an amino acid sequence occurring in type II collagen from vertebrates, in particular from fish, amphibians, reptiles, birds and mammals, in particular in pig, sheep, cattle, rodent, kangaroo, horse or from invertebrates, in particular jellyfish, in particular an amino acid sequence occurring in type II collagen from cattle.

Particularly preferably, the type II collagen, in particular type II collagen peptide, comprises the amino acid sequence according to SEQ ID No. 2. Preferably, the type II collagen, in particular type II collagen peptide, consists of the amino acid sequence according to SEQ ID No. 2.

In another preferred embodiment of the present invention, the type II collagen, in particular type II collagen peptide, has at least 80%, preferably at least 85%, preferably at least 90%, preferably at least 95%, preferably at least 96%, preferably at least 97%, preferably at least 98%, preferably at least 99%, sequence identity with the amino acid sequence according to SEQ ID No. 2.

According to a further preferred embodiment of the present invention, the type II collagen, in particular type II collagen peptide, comprises the amino acid sequence according to SEQ ID No. 4. Preferably, the type II collagen, in particular type II collagen peptide, consists of the amino acid sequence according to SEQ ID No. 4.

In another preferred embodiment of the present invention, the type II collagen, in particular type II collagen peptide, has at least 80%, preferably at least 85%, preferably at least 90%, preferably at least 95%, preferably at least 96%, preferably at least 97%, preferably at least 98%, preferably at least 99%, sequence identity with the amino acid sequence according to SEQ ID No. 4.

Preferably, the amino acid sequence of the recombinant type II collagen, in particular type II collagen peptide, is the amino acid sequence of a naturally occurring type II collagen, in particular type II collagen peptide. Preferably, the amino acid sequence of the recombinant type II collagen, in particular type II collagen peptide, is the amino acid sequence of a naturally non-occurring type II collagen, in particular type II collagen peptide. Preferably, the amino acid sequence of the recombinant type II collagen, in particular type II collagen peptide, is the amino acid sequence of a genetically modified type II collagen, in particular type II collagen peptide.

Particularly preferably, the type II collagen according to the invention, in particular type II collagen peptide, has an amino acid sequence found in non-human collagen, in particular in non-human type II collagen peptides, preferably in the al chain of non-human type II collagen, in particular an amino acid sequence occurring in bovine, porcine, equine, ovine, piscine or avian collagen, in particular an amino acid sequence occurring in bovine collagen.

In a preferred embodiment of the present invention, the recombinant type II collagen, in particular the recombinant type II collagen peptide, is resistant to collagenase, in particular resistant to digestion by human collagenases.

According to a further preferred embodiment of the present invention, the recombinant type II collagen, in particular type II collagen peptide, is capable of inducing oral tolerance, in particular to endogenously present collagen, in particular endogenously present type II collagen, in particular endogenous type II collagen present in cartilage tissue.

According to a further preferred embodiment of the present invention, the recombinant type II collagen, in particular type II collagen peptide, is capable of suppressing the synthesis of immunoglobulins.

According to another preferred embodiment of the present invention, the recombinant type II collagen, in particular type II collagen peptide, is capable of suppressing the synthesis of pro-inflammatory cytokines.

According to another preferred embodiment of the present invention, the recombinant type II collagen, in particular type II collagen peptide, is capable of stimulating the synthesis of anti-inflammatory cytokines.

According to another preferred embodiment of the present invention, the recombinant type II collagen, in particular type II collagen peptide, is capable of suppressing the synthesis of immunoglobulins, suppressing the synthesis of pro-inflammatory cytokines and stimulating the synthesis of anti-inflammatory cytokines. According to a preferred embodiment of the present invention, the recombinant type II collagen, in particular type II collagen peptide, for use in a therapeutic method for oral therapy of cartilage diseases of a human or animal patient has a molecular weight in a range from 5 to 400 kDa, in particular 10 to 390 kDa, in particular 10 to 350 kDa, in particular 10 to 300 kDa, in particular 10 to 110 kDa, in particular 40 to 110 kDa, in particular 40 to 100 kDa, in particular 11 to 105 kDa, in particular 15 to 100 kDa, in particular 20 to 99 kDa, in particular 25 to 95 kDa, in particular 30 to 95 kDa, in particular 35 to 95 kDa. Preferably, the recombinant type II collagen has a molecular weight in a range from 40 to 50 kDa, in particular 45 kDa.

In a preferred embodiment of the present invention, the recombinant type II collagen, in particular type II collagen peptide, according to the invention is used alone, i.e. in isolated form, i.e. without further substances, in the use provided for in the invention.

In a preferred embodiment of the present invention, the recombinant type II collagen, in particular type II collagen peptide, according to the invention is present as a homogeneous preparation, in particular as a homogeneous preparation of a single recombinant type II collagen, in particular type II collagen peptide, with a uniform molecular weight.

In a further embodiment of the present invention, the recombinant type II collagen, in particular type II collagen peptide, according to the invention is used as the sole substance having biological efficiency in the use provided for in the invention.

In a particularly preferred embodiment, the present invention relates to compositions comprising at least one recombinant type II collagen, in particular at least one recombinant type II collagen peptide, which compositions do not contain any substances other than the at least one recombinant type II collagen, in particular the at least one recombinant type II collagen peptide, and optionally a pharmaceutically acceptable and/or food-compatible carrier.

In a particularly preferred embodiment, the composition having at least one recombinant type II collagen, in particular at least one recombinant type II collagen peptide, is present in a dosage form suitable for oral administration in a human or animal body.

The present invention also relates to a composition comprising at least one recombinant type II collagen, in particular at least one recombinant type II collagen peptide, according to the present invention and at least one pharmaceutically acceptable and/or food-acceptable carrier and, optionally, at least one additive or excipient for use in a therapeutic method for oral therapy of cartilage diseases.

The present invention therefore also relates to a composition for use in a method for the therapeutic prophylaxis or therapeutic treatment of immune intolerance reactions to type II collagen, in particular endogenous type II collagen, by the induction of oral tolerance to type II collagen, in particular endogenous type II collagen.

The present invention also relates to a composition for use in a method of inducing oral tolerance to type II collagen, in particular endogenous type II collagen, wherein the composition results in the induction of oral tolerance in the human or animal body.

The present invention also relates to a composition comprising recombinant type II collagen, in particular recombinant type II collagen peptides, in particular pharmaceutical compositions or food supplements, or food or luxury food products, for use in inducing oral tolerance to type II collagen, in particular endogenous type II collagen.

Compositions according to the invention for oral administration may be, in particular, pharmaceutical compositions, food supplements or food or luxury food products. In particular, the compositions according to the invention are pharmaceutical compositions. In particular, the compositions according to the invention are food supplements.

In particular, the present invention relates to a pharmaceutical composition comprising a type II collagen, in particular type II collagen peptide, according to the invention, and at least one pharmaceutically acceptable carrier, and to the pharmaceutical composition for use in a method for therapeutic treatment of cartilage diseases of the human or animal body. Accordingly, it may be envisaged to administer the type II collagen, in particular type II collagen peptide, according to the invention in the form of a pharmaceutical composition. Particularly advantageously, the pharmaceutical composition according to the invention is administered, for example, in the form of tablets, lozenges, chewable tablets, powder, granules, hard capsule, soft capsule, capsule, bite capsules, dragees, pastilles, extrudate, juices, suspension, gels or ointments.

In a particularly preferred embodiment of the present invention, the type II collagen used according to the invention, in particular type II collagen peptide, is present in a dosage form allowing a prolonged intestinal release, in particular a prolonged release capsule.

In a particularly preferred embodiment, the composition according to the invention is present in a form suitable for oral administration, in particular with a dose of 1 to 60 mg/day, in particular 5 to 50 mg/day, of recombinant type II collagen.

The present invention further relates to a food supplement comprising a type II collagen according to the invention, in particular type II collagen peptide, and at least one food-acceptable carrier, as well as the food supplement for use in a method for therapeutic treatment of cartilage diseases of the human or animal body. Accordingly, it may be envisaged to administer the type II collagen according to the invention, in particular type II collagen peptide, in the form of a food supplement.

Particularly advantageously, the food supplement according to the invention is present as a hard capsule, soft capsule, capsule, bite capsule, tablet, dragee, pastille, sachet, extrudate, solution, suspension or gel, for example in an ampoule, as granules or powder.

It is also an object of the invention to provide a food or luxury food product comprising a type II collagen, in particular type II collagen peptide, according to the invention, and the food or luxury food product for use in a method for the therapeutic treatment of cartilage diseases of the human or animal body. According to a preferred embodiment, the food or luxury food product is a chocolate bar, protein bar, cereal bar, instant powder for preparing beverages, milk, dairy products, for example yoghurt, whey or curd and milk substitutes, for example soy milk, rice milk, almond milk and coconut milk, functional food or a beverage, for example refreshment or fitness drink.

Provided that the recombinant type II collagen, in particular type II collagen peptide, according to a preferred embodiment of the invention is not to be used as the sole ingredient having a biological effectiveness of a composition, in particular of a pharmaceutical composition, of a food supplement, or of a food or luxury product, it may be combined with one or more further additives or excipients, in particular those having a positive effect on general health, in particular on cartilage health and/or endurance performance. Preferred excipients according to the invention are selected from the group consisting of vitamin C, vitamins of the B, D, E and K series, omega-3 fatty acids, omega-6 fatty acids, conjugated linoleic acids, caffeine and derivatives thereof, guarana extract, rosehip extract, green tea extract, epigallocatechin gallate, creatine, L-carnitine, α-lipoic acid, N-acetylcysteine, NADH, D-ribose, magnesium aspartate, antioxidants such as anthocyanins, carotenoids, flavonoids, resveratrol, glutathione and superoxide dismutase, minerals such as iron, magnesium, calcium, zinc, selenium and phosphorus, and other proteins, hydrolysates and peptides such as soya, wheat and whey protein.

In a particularly preferred embodiment, it may be provided that the composition according to the invention, in particular the pharmaceutical composition, the food supplement or the food product, or luxury food product, has an excipient, for example chondroitin, chondroitin sulphate, hyaluronic acid, aflapin, Univestin 5-Glocsin, glucosamine, glucosamine sulphate and/or methylsulphonylmethane (MSM).

In a further preferred embodiment, it may be provided that the composition according to the invention, in particular the pharmaceutical composition, the food supplement or a food product, or luxury food product, has an additive, wherein the additive may be a recombinantly produced collagen hydrolysate, a collagen hydrolysate originated from natural sources, a recombinantly produced type I collagen, a type I collagen obtained from natural sources, or a combination thereof.

In a further preferred embodiment of the present invention, the products according to the invention, in particular the pharmaceutical composition, the food supplement, or the food or luxury food product, do not contain any other proteins or peptides, in particular no other collagen peptides, in addition to the type II collagen, in particular type II collagen peptide, according to the invention.

The present invention also relates to methods for therapy, in particular for the prevention and/or treatment of cartilage diseases, according to which an amount of at least one of the recombinant type II collagens according to the invention, in particular type II collagen peptides, optionally with a carrier and, optionally, an excipient or additive, is administered orally to the human or animal body in an amount sufficient for the therapeutic purpose.

The present invention also relates to methods for inducing oral tolerance to type II collagen, in particular endogenous type II collagen, in a human or animal body, comprising the administration of a sufficient amount for a therapeutic purpose of at least one of the recombinant type II collagens according to the invention, in particular recombinant type II collagen peptides, optionally by means of a carrier and, optionally, an excipient or additive, wherein the administration is oral.

The present invention also relates to methods for the therapeutic treatment or therapeutic prophylaxis of immune intolerance to type II collagen, in particular type II collagen peptide, comprising the oral administration of a sufficient amount for a therapeutic purpose of at least one of the recombinant type II collagens according to the invention, in particular recombinant type II collagen peptides, optionally by means of a carrier and, optionally, an excipient or additive.

The present invention also relates to the use of recombinant type II collagen, in particular recombinant type II collagen peptides, in non-therapeutic methods for maintaining cartilage health of a human or animal, according to which an amount of at least one of the recombinant type II collagens according to the invention, in particular type II collagen peptides, optionally by means of a carrier and, optionally, an excipient or additive, is orally administered to the human or animal body sufficient for maintaining cartilage health. In this particularly preferred embodiment of the present invention, the human or animal has no cartilage disease present. Accordingly, in a particularly preferred embodiment, oral administration of recombinant type II collagen, in particular recombinant type II collagen peptides, to a human or animal may be provided which does not have cartilage disease and maintains its cartilage health by administration of the recombinant type II collagen, in particular recombinant type II collagen peptide.

In addition, the present invention relates to a method for producing a recombinant type II collagen, in particular type II collagen peptide, which can be used according to the invention, comprising the method steps:
a) providing an expression system having at least one expression cassette, wherein the expression cassette has at least one nucleotide sequence encoding a type II collagen, in particular type II collagen peptide,
b) cultivating the expression system under conditions which allow expression of the type II collagen, in particular type II collagen peptide, and
c) obtaining the type II collagen, in particular type II collagen peptide, according to the invention.

The method provided according to the invention for producing a recombinant type II collagen, in particular type II collagen peptide, which can be used according to the invention, is characterised in particular by the fact that a precisely defined, recombinantly produced type II collagen, in particular type II collagen peptide, is obtained which, in particular on account of its biological effectiveness, is suitable for use in a method for the therapeutic treatment of cartilage diseases of the human or animal body or for maintaining cartilage health.

The type II collagen, in particular type II collagen peptide, provided according to the invention has a particularly high purity due to its recombinant production method compared to type II collagen, in particular type II collagen peptides, obtained hydrolytically from natural sources. It can be provided in a wide variety of expression systems, even on an industrial scale, without undesirable contamination, wherein the type II collagen according to the invention, in particular type II collagen peptide, advantageously has biological effectiveness at the same time.

Recombinant type II collagen and its production is described, for example, in U.S. Pat. No. 5,593,859. This document discloses the obtaining of recombinant type II collagen peptides as well as hydroxylation and fibrillogenesis for the obtaining of procollagen in recombinant cell culture and is fully included in the present disclosure with respect to the production of recombinant type II collagen as well as recombinant type II collagen peptides, in particular also in hydroxylated and triple-helical form.

In a preferred embodiment, the biological effectiveness of the type II collagen found according to the invention, in particular of the type II collagen peptides, and associated therewith its or their suitability for use in a method for the therapeutic treatment of cartilage diseases of the human or animal body, is advantageously already conferred on the type II collagen obtained directly from the method according to the invention, in particular type II collagen peptides, without the need for further processing steps. Thus, in a preferred embodiment, both the hydroxylated and the non-hydroxylated type II collagens, in particular type II collagen peptides, according to the present invention have a biological effectiveness, in particular at least the same biological effectiveness as type II collagen obtained from natural sources, particularly preferably a better biological effectiveness than type II collagen obtained from natural sources.

Particularly advantageous in this respect is that the type II collagens according to the invention, in particular type II collagen peptides, surprisingly have biological effectiveness even in non-hydroxylated form, preferably the same biological effectiveness as type II collagen obtained from natural sources, particularly preferably better biological effectiveness than type II collagen obtained from natural sources.

Preferably, both the hydroxylated and the non-hydroxylated type II collagens, in particular type II collagen peptides, according to the present invention exhibit biological effectiveness, preferably at least the same biological effectiveness as type II collagen obtained from natural sources, more preferably better biological effectiveness than type II collagen obtained from natural sources.

Preferably, the expression system provided in step a) is a host cell, in particular a prokaryotic or eukaryotic cell.

Preferably, the expression system is a host cell selected from the group consisting of bacterial cell, yeast cell, fungal cell, mammalian cell, insect cell and plant cell.

Preferably, the expression system, in particular the host cell, is a bacterial cell, in particular of the species Escherichia coli or Bacillus subtilis.

In a further preferred embodiment, the expression system, in particular the host cell, is a yeast cell, in particular of the species Saccharomyces cerevisiae, Pichia pastoris or Ogataea angusta (Hansenula polymorpha), in particular Pichia pastoris.

Preferably, the expression system, in particular the host cell, is a fungal cell, in particular of the species Aspergillus niger.

In another preferred embodiment of the present invention, the expression system, in particular the host cell, is a mammalian cell, in particular a CHO cell, a HeLa cell or a HEK293 cell.

Preferably, the expression system, in particular the host cell, is an insect cell, in particular an Sf-9, Sf-21 or Tn-5 cell.

Preferably, the expression system, in particular the host cell, is a plant cell, in particular a maize or tobacco cell.

In another preferred embodiment of the present invention, the expression system provided in step a) is a host cell capable of hydroxylating proline, lysine or proline and lysine residues of the expressed collagen peptide. Preferably, the expression system provided in step a) is a host cell capable of hydroxylating proline, lysine or proline and lysine residues of the expressed collagen peptide.

Preferably, the expression system provided in step a), is an expression system having prolyl hydroxylase and/or lysyl hydroxylase activity. Preferably, the expression system provided in step a) is a host cell having prolyl hydroxylase and/or lysyl hydroxylase activity.

In a preferred embodiment, the expression system provided in step a) is a host cell that has at least one expression cassette comprising a prolyl 4-hydroxylase-encoding polynucleotide sequence.

Particularly preferably, the expression system provided in step a) is a host cell that has at least one expression cassette comprising a prolyl-4-hydroxylase-encoding polynucleotide sequence, so that in method step c) an in vivo hydroxylated type II collagen, in particular type II collagen peptide, is obtained.

In a preferred embodiment, the expression system provided in step a) is a host cell comprising at least one expression cassette comprising a lysyl hydroxylase-encoding polynucleotide sequence. Particularly preferably, the expression system provided in step a) is a host cell that has at least one expression cassette comprising a lysyl hydroxylase-encoding polynucleotide sequence, so that in method step c) an in vivo hydroxylated type II collagen, in particular type II collagen peptide, is obtained.

In further preferred embodiment of the present invention, the expression system provided in step a) is a host cell that has at least one expression cassette comprising a prolyl 4-hydroxylase-encoding polynucleotide sequence and at least one expression cassette comprising a lysyl hydroxylase-encoding polynucleotide sequence. Particularly preferably, the expression system provided in step a) is a host cell that comprises at least one expression cassette comprising a prolyl 4-hydroxylase-encoding polynucleotide sequence and at least one expression cassette comprising a lysyl hydroxylase-encoding polynucleotide sequence, such that in method step c) an in vivo hydroxylated type II collagen, in particular type II collagen peptide, is obtained.

In a preferred embodiment of the present invention, the prolyl 4-hydroxylase-encoding polynucleotide sequence comprises the nucleotide sequence according to SEQ ID No. 5. Particularly preferably, the prolyl-4-hydroxylase-encoding polynucleotide sequence encodes a monomeric prolyl-4-hydroxylase, in particular a prolyl-4-hydroxylase having an amino acid sequence according to SEQ ID No. 6, preferably consisting of an amino acid sequence according to SEQ ID No. 6.

The present invention thus also relates to a method for producing a recombinant type II collagen, in particular type II collagen peptide, which can be used according to the invention, in particular an in vivo hydroxylated type II collagen, in particular type II collagen peptide, comprising the method steps of
a) providing an expression system having at least one expression cassette, wherein the expression cassette has at least one nucleotide sequence encoding a type II collagen, in particular type II collagen peptide, and wherein the expression system is capable of hydroxylating proline, lysine or proline and lysine residues of the expressed collagen peptide
b) cultivating the expression system under conditions which allow expression and hydroxylation of the type II collagen, in particular type II collagen peptide,
c) obtaining the type II collagen, in particular type II collagen peptide, according to the invention, in particular the in vivo hydroxylated type II collagen, in particular type II collagen peptide.

By means of the aforementioned method, it is thus advantageously possible to obtain an in vivo hydroxylated recombinant type II collagen, in particular type II collagen peptide, which is characterised by a specific pattern of post-translational modifications, in particular hydroxylations and glycosylations, depending on the cell-based expression system used. In this way, it is advantageously possible in particular to obtain directly, i.e. without the need for subsequent modification, a type II collagen, in particular a recombinantly produced type II collagen peptide, with desired biological effectiveness for use in a method for the therapeutic treatment of cartilage diseases of the human or animal body.

In a preferred embodiment, the recombinant in vivo hydroxylated collagen peptide produced according to the present invention has biological effectiveness. According to a further embodiment of the present invention, the expression system provided in step a) is an expression system that is not capable of causing hydroxylation of proline, lysine or proline and lysine residues of the expressed collagen peptide, in particular the expression system provided in step a) does not have prolyl hydroxylase and lysyl hydroxylase activity.

Thus, the present invention comprises a method for producing a recombinant collagen peptide usable according to the invention, in particular a non-hydroxylated collagen peptide, comprising the method steps of
a) providing an expression system having at least one expression cassette, wherein the expression cassette has at least one nucleotide sequence encoding a type II collagen, in particular type II collagen peptide, and wherein the expression system is not capable of hydroxylating proline, lysine or proline and lysine residues of the expressed type II collagen, in particular type II collagen peptide,
b) cultivating the expression system under conditions which allow expression of the type II collagen, in particular type II collagen peptide,
c) obtaining the type II collagen, in particular type II collagen peptide, in particular the non-hydroxylated type II collagen, in particular type II collagen peptide, according to the invention.

According to a preferred embodiment of the present invention, the at least one nucleotide sequence of the at least one expression cassette is codon-optimised, i.e. those codons in the nucleotide sequence which are not or not preferably used by the translation system of the provided expression system, in particular the provided cell-based expression system, in particular the provided host cell, are replaced by codons which are preferably used by the translation system of the provided expression system, in particular of the provided cell-based expression system, in particular of the provided host cell, without thereby changing the amino acid sequence of the encoded peptide or protein.

In a preferred embodiment of the present invention, the type II collagen, in particular type II collagen peptide, encoded by the nucleotide sequence is a type II collagen, in particular type II collagen peptide, of a vertebrate animal, in particular a mammal, for example of man or of a non-human mammal, for example horse, kangaroo, rodent, pig, sheep or cattle, of a bird, for example chicken, of a fish, of an amphibian, of a reptile or of an invertebrate, for example jellyfish.

In a preferred embodiment of the present invention, the expression cassette provided in step a) comprises at least one nucleotide sequence according to SEQ ID No. 1.

Particularly preferably, the expression cassette provided in step a) comprises at least one nucleotide sequence having a sequence identity of at least 90%, preferably at least 95%, preferably at least 96%, preferably at least 97%, preferably at least 98%, preferably at least 99%, to the nucleotide sequence according to SEQ ID No. 1.

Particularly preferably, the type II collagen, in particular type II collagen peptide, encoded by the nucleotide sequence is a type II collagen, in particular type II collagen peptide, comprising the amino acid sequence according to SEQ ID No. 2. Preferably, the type II collagen, in particular type II collagen peptide, encoded by the nucleotide sequence consists of the amino acid sequence according to SEQ ID No. 2.

In a further preferred embodiment of the present invention, the type II collagen, in particular type II collagen peptide, encoded by the nucleotide sequence has at least 80%, preferably at least 85%, preferably at least 90%, preferably at least 95%, preferably at least 96%, preferably at least 97%, preferably at least 98%, preferably at least 99%, sequence identity with the amino acid sequence according to SEQ ID No. 2.

According to a preferred embodiment of the present invention, the expression cassette provided in step a) comprises at least one nucleotide sequence according to SEQ ID No. 3.

Particularly preferably, the expression cassette provided in step a) comprises at least one nucleotide sequence with a sequence identity of at least 90%, preferably at least 95%, preferably at least 96%, preferably at least 97%, preferably at least 98%, preferably at least 99%, to the nucleotide sequence according to SEQ ID No. 3.

Particularly preferably, the type II collagen, in particular type II collagen peptide, encoded by the nucleotide sequence is a type II collagen, in particular type II collagen peptide, comprising the amino acid sequence according to SEQ ID No. 4. Preferably, the type II collagen, in particular type II collagen peptide, encoded by the nucleotide sequence consists of the amino acid sequence according to SEQ ID No. 4.

In a further preferred embodiment of the present invention, the type II collagen, in particular type II collagen peptide, encoded by the nucleotide sequence has at least 80%, preferably at least 85%, preferably at least 90%, preferably at least 95%, preferably at least 96%, preferably at least 97%, preferably at least 98%, preferably at least 99%, sequence identity with the amino acid sequence according to SEQ ID No. 4.

Preferably, the type II collagen, in particular type II collagen peptide, encoded by the nucleotide sequence is a naturally occurring type II collagen, in particular type II collagen peptide. In a further preferred embodiment of the present invention, the type II collagen, in particular type II collagen peptide, encoded by the nucleotide sequence is not a naturally occurring type II collagen, in particular type II collagen peptide. Preferably, the type II collagen, in particular type II collagen peptide, encoded by the nucleotide sequence is a genetically modified collagen peptide.

According to a preferred embodiment of the present invention, the at least one nucleotide sequence encodes a type II collagen peptide with a molecular weight in a range from 5 to 400 kDa, in particular 10 to 390 kDa, in particular 10 to 350 kDa, in particular 10 to 300 kDa, in particular 10 to 110 kDa, in particular 40 to 110 kDa, in particular 40 to 100 kDa, in particular 11 to 105 kDa, in particular 15 to 100 kDa, in particular 20 to 99 kDa, in particular 25 to 95 kDa, in particular 30 to 95 kDa, in particular 35 to 95 kDa, in particular from 40 to 50 kDa, in particular 45 kDa.

In a particularly preferred embodiment of the present invention, the methods according to the invention are distinguished in that in method step b) conditions are selected which allow the formation of a non-denatured, i.e. native, type II collagen, in particular type II collagen peptide.

In a particularly preferred embodiment of the present invention, the methods according to the invention are characterised in that in method step b) conditions are selected which allow the formation of a triple-helical form of the type II collagen, in particular type II collagen peptide.

In a particularly preferred embodiment, the methods of the invention may lead to the production of homogeneous and isolated preparations of specific type II collagen peptides with a uniform molecular weight.

In a particularly preferred embodiment, the invention provides for also providing mixtures of such produced isolated and homogeneous preparations of type II collagen peptides, each with a uniform molecular weight.

The invention also provides for the preparation of recombinant collagen peptide hydrolysates by lysis, in particular hydrolysis, from type II collagen peptides with a uniform molecular weight, which may be produced homogeneously and isolated by means of the methods according to the invention. In particular, the present invention provides both the homogeneously isolated type II collagen peptides with uniform molecular weight, mixtures thereof or hydrolysates thereof for oral therapy of cartilage diseases of a human or animal body provided according to the invention.

According to the present invention, the methods according to the invention are characterised in that, following method step b) or c), in method step d), a recombinant type II collagen peptide hydrolysate is obtained by lysis, in particular hydrolysis, of the expressed type II collagen, in particular type II collagen peptide.

The type II collagen peptide hydrolysate obtained according to the invention by method step d) can be used as a recombinant type II collagen peptide according to the invention either in the form of this type II collagen hydrolysate or after isolation of one or more type II collagen peptides, which are preferably then present homogeneously and in isolation.

In a particularly preferred embodiment of the present invention, it may also be provided that homogeneous and isolated type II collagen peptides present are mixed with each other and thus constitute a mixture of recombinant type II collagen peptides.

In a particularly preferred embodiment, the invention therefore also relates to a recombinant type II collagen peptide which is present in isolated homogeneous form having a uniform molecular weight, or as a recombinant type II collagen peptide which is present in a mixture with recombinant or natural, in particular recombinant type II collagen peptides, or in a hydrolysate of a recombinant type II collagen, in particular recombinant type II collagen peptide.

The nucleotide sequences encoding the recombinant type II collagen peptide which can be used according to the invention can be obtained in a customary manner, as described for example in WO 93/07889, US 2006/0147501, U.S. Pat. No. 5,593,859 or US 2008/0081353.

In a preferred embodiment of the present invention, the type II collagen, in particular type II collagen peptide, which can be used according to the invention, preferably produced by one of the aforementioned methods according to the invention, for use in a method for therapeutic treatment of cartilage diseases of the human or animal body is a non-hydroxylated, partially hydroxylated or fully hydroxylated type II collagen peptide, preferably a non-hydroxylated type II collagen peptide, preferably a partially hydroxylated type II collagen peptide, preferably a fully hydroxylated type II collagen peptide.

In a preferred embodiment of the present invention, the type II collagen, in particular recombinant type II collagen peptide, which can be used according to the invention, preferably produced by one of the above methods, for use in a method for therapeutic treatment of cartilage diseases of the human or animal body is a glycosylated collagen peptide. Preferably, the type II collagen, in particular type II collagen peptide is glycosylated in vivo, preferably glycosylated ex vivo.

In a further preferred embodiment of the present invention, the type II collagen, in particular type II collagen peptide, which can be used according to the invention, preferably produced by one of the methods according to the invention, is a non-glycosylated type II collagen, in particular type II collagen peptide.

According to the invention, the term “biological effectiveness” is preferably understood to mean the ability of the type II collagens, in particular type II collagen peptides, which can be used according to the invention, to modulate the immune system, in particular to suppress the synthesis of immunoglobulins, in particular IgE, IgA, IgM and/or IgG.

According to the invention, the term “biological effectiveness” is also preferably understood to mean the ability of the type II collagens, in particular type II collagen peptides, which can be used according to the invention, to suppress the formation and activity of pro-inflammatory cytokines, in particular TNFα, IL-6 and IFNγ, or to stimulate the synthesis and activity of anti-inflammatory cytokines, in particular IL-4, IL-10 and TGF-ß1, in particular both.

According to the invention “biological effectiveness” is preferably understood to mean that the type II collagens in particular type II collagen peptides, which can be used according to the invention, are capable of immune-modulation, in particular of suppression of the synthesis of immunoglobulins, in particular IgE, IgA, IgM and/or IgG, to suppress the formation of pro-inflammatory cytokines, in particular TNFα, IL-6 and IFNγ, and to stimulate the synthesis of anti-inflammatory cytokines, in particular IL-4, IL-10 and TGF-ß1.

In a particularly preferred embodiment, the biological effectiveness is determined in particular by means of detection methods familiar to the skilled person for immune-modulating, in particular stimulating as well as suppressing activities of substances and for anti-inflammatory cytokines and pro-inflammatory cytokines. In particular, the biological effectiveness within the meaning of the present invention is determined by means of the method according to Example 2, Example 3, Example 4 and/or Example 5. According to the invention, the term “biological effectiveness” is preferably also understood to mean the ability of the type II collagens, in particular type II collagen peptides, which can be used according to the invention, to induce oral tolerance. In a particularly preferred embodiment, the presence of oral tolerance is determined by means of detection methods familiar to the skilled person for determining the ability of a substance to induce oral tolerance, in particular by means of the method according to Example 2, Example 3, Example 4 and/or Example 5.

In the context of the present invention, pro-inflammatory cytokines are in particular TNFα, IL-6 and IFN-gamma.

In the context of the present invention, anti-inflammatory cytokines are in particular IL-4, IL-10 and TGF-ß1.

In the context of the present invention, the term “suppression” is understood to mean the partial or complete suppression of a synthesis of proteins, which may present itself in particular as a reduction or inhibition of protein synthesis or as a reduction or inhibition of mRNA synthesis affecting the proteins.

In the context of the present invention, the term “collagen” is understood in a manner customary in the art, in particular as defined for example in WO 01/34646. In a further preferred embodiment, the term “collagen” is understood to mean a collagen protein or peptide having the sequence glycine-proline, glycine-4-hydroxyproline or glycine-X-4-hydroxyproline, preferably the repetitive motif (Gly-X—Y)n, wherein X and Y can be any amino acid, preferably proline and 4-hydroxyproline. Particularly preferably, the term “collagen” is understood to mean a peptide having the repetitive motif (Gly-Pro-Y)n and/or (Gly-X-Hyp)m, wherein X and Y can be any amino acid.

A “type II collagen” according to the present invention is a collagen as understood in customary manner according to the foregoing, wherein the type II collagen has the amino acid sequence of a naturally occurring type II collagen, in particular the amino acid sequence of a type II collagen of a vertebrate, in particular pig, sheep, cattle, rodent, horse, bird, fish, reptile or amphibian or of an invertebrate, in particular jellyfish.

The type II collagen may be present as a monomeric collagen peptide, also referred to herein as a single-stranded collagen peptide, or as a di- or trimer, in particular a trimer, having at least two, in particular three collagen peptides, in particular the same single-stranded collagen peptides. In particular, the type II collagen may be present as a triple-helical type II collagen peptide, in particular native type II collagen.

In the context of the present invention, the term “type II collagen peptide” is understood to mean a single-stranded type II collagen peptide having an amino acid sequence present in type II collagen according to the foregoing definition, wherein the peptide is an oligopeptide or polypeptide. In particular, the type II collagen peptide may be present in chemically modified form, in particular hydroxylated and/or glycosylated form, or may be unmodified.

Preferably, the recombinant type II collagen, in particular type II collagen peptide, used according to the invention can have a sequence modification, in particular a function-preserving sequence modification of a naturally occurring type II collagen, in particular type II collagen peptide.

Accordingly, a “type II collagen” is also understood to mean a function-preserving sequence modification of a naturally occurring type II collagen, in particular type II collagen peptide, in particular if these have, at amino acid level, an amino acid sequence identity of at least 80% to the amino acid sequence of the naturally occurring type II collagen, in particular at least 85%, in particular at least 90%, in particular at least 95%, in particular at least 96%, in particular at least 97%, in particular at least 98%, in particular at least 99% amino acid sequence identity. According to the invention, a type II collagen is present if the recombinant type II collagen either has exactly the amino acid sequence that occurs in a naturally occurring type II collagen or if a functionally conserved sequence modification with an amino acid sequence identity of at least 80%, at least 85%, at least 90%, at least 91%, at least 92%, at least 93%, at least 94%, at least 95%, at least 96%, at least 97%, at least 98%, at least 99% with respect to a naturally occurring type II collagen, in particular with respect to naturally occurring type II collagen from a vertebrate, in particular pig, sheep, cattle, rodent, kangaroo, horse, bird, reptile, amphibian or fish or invertebrate, in particular jellyfish, in particular when this amino acid identity is present with respect to a naturally occurring type II collagen amino acid sequence from cattle.

In the context of the present invention, the amino acid sequence identity is determined using the Smith-Waterman algorithm (SSE2, Michael Farrar, 2006, 7.2 Nov. 2010) with the parameters BL50 matrix (15:−5), Open/ext: −12/−2.

According to the invention, the term “function-preserving sequence modification” is understood to mean the modification of a given, in particular naturally occurring, amino acid sequence, in particular the replacement, addition and/or deletion of individual or several amino acids, which results in an amino acid sequence which deviates from the given amino acid sequence, but the modified amino acid sequence retains the function characteristic of the given amino acid sequence, in particular its biological effectiveness.

Preferably, a “function-preserving sequence modification” is understood to mean a modification of a given amino acid sequence, in particular a naturally occurring amino acid sequence, in which the function characteristic of the given amino acid sequence, in particular a biological effectiveness thereof, is retained to at least 50%, preferably at least 60%, preferably at least 70%, preferably at least 80%, preferably at least 90%, preferably at least 95%, preferably 100%. Further preferably, according to the invention, a “function-preserving sequence modification” is understood to mean a modification of a given amino acid sequence in which the modified amino acid sequence has at least 50%, preferably at least 55%, preferably at least 60%, preferably at least 65%, preferably at least 70%, preferably at least 75%, preferably at least 80%, preferably at least 85%, preferably at least 90%, sequence homology to the given amino acid sequence.

Particularly preferably, a sequence modification, in particular a “function-preserving sequence modification” in the context of the present invention is a modification of a given, in particular naturally occurring amino acid sequence, in which one or more amino acids with specific chemical-physical properties have been replaced by one or more amino acids with in each case the same or similar chemical-physical properties, in particular, for example, an amino acid with a non-polar side chain (for example, Ala, Val, Met, Leu, Ile, Pro, Trp, Phe) by another amino acid with a non-polar side chain (for example, Ala, Val, Met, Leu, Ile, Pro, Trp, Phe), an amino acid with a polar neural side chain (for example Tyr, Thr, Gln, Gly, Ser, Cys, Asn) by another amino acid with a polar neural side chain (for example Tyr, Thr, Gln, Gly, Ser, Cys, Asn), an amino acid with an acid side chain (for example Glu, Asp) by another amino acid with an acidic side chain (for example Glu, Asp) and/or an amino acid with a basic side chain (for example Lys, Arg, His) by another amino acid with a basic side chain (for example Lys, Arg, His). According to this embodiment, the chemical-physical properties of a given amino acid sequence are maintained in the “function-preserving sequence conversion” or change only slightly.

In a further embodiment, it may be provided that the sequence modification, in particular a “function-preserving sequence modification”, consists in replacing at least one amino acid of a given amino acid sequence, in particular a naturally occurring amino acid sequence, preferably at least one non-essential amino acid, in particular Ala, Asn, Asp, Glu, Ser, of the given amino acid sequence, in particular the naturally occurring amino acid sequence, by at least one very specific amino acid, in particular at least one essential amino acid, in particular Ile, Leu, Lys, Met, Phe, Thr. Trp, Val, His, Cys, Tyr, particularly preferably Trp, wherein the characteristic function of the given amino acid sequence, in particular the naturally occurring amino acid sequence, in particular the biological effectiveness, in particular the biological effectiveness according to the present invention, in particular according to the detection shown in Example 2. Example 3, Example 4 and/or Example 5, is retained.

According to the present invention, a sequence modification, in particular a “function-preserving sequence modification” is also understood to mean the modification of a given amino acid sequence, in particular a naturally occurring amino acid sequence, which consists in adding to the given amino acid sequence, in particular the naturally occurring amino acid sequence, at least one amino acid, preferably at least one essential amino acid, in particular Ile, Leu, Lys, Met, Phe, Thr, Trp, Val, His, Cys, Tyr, particularly preferably Trp, wherein the characteristic function of the given amino acid sequence, in particular the naturally occurring amino acid sequence, in particular the biological effectiveness according to the present invention, in particular the biological effectiveness according to the detection shown in Example 2, Example 3, Example 4 and/or Example 5, is retained. In this regard, it may be provided according to the invention to add the at least one amino acid, preferably the at least one essential amino acid, in particular Ile, Leu, Lys, Met, Phe, Thr, Trp, Val, His, Cys, Tyr, particularly preferably Trp, N-terminally, C-terminally and/or within the amino acid sequence.

In the context of the present invention, the term “amino acid modification” means a chemical change, if any, of one or more amino acids before, after or during the synthesis of the recombinant type II collagen, in particular type II collagen peptide, while retaining the original amino acid backbone, in particular one or more proteinogenic amino acids, of the type II collagen peptide. Thus, the term comprises both the use of chemically modified amino acids for the synthesis of the type II collagen, in particular type II collagen peptide, according to the invention and the chemical modification of the amino acids after or during the synthesis of the type II collagen, in particular type II collagen peptide. Amino acid modifications typical for collagen peptides are in particular hydroxylations at proline and lysine residues as well as glycosylations of hydroxylated lysine residues. However, according to the invention, the term also comprises other chemical modifications of amino acids, such as phosphorylations, N-glycosylations, acetylations, methylations or myristoylations.

In the context of the present invention, a recombinant type II collagen, in particular type II collagen peptide, or a recombinantly produced type II collagen, in particular type II collagen peptide, is understood to mean a type II collagen, in particular type II collagen peptide, obtained by biotechnological recombinant production using an expression system. According to the invention, the recombinant type II collagen, in particular type II collagen peptide, or the recombinantly produced type II collagen, in particular type II collagen peptide, has in common that they are not obtained from natural sources.

In a particularly preferred embodiment of the present invention, the recombinant type II collagen, in particular type II collagen peptide, is present in the form of a homogeneous preparation of this type II collagen or type II collagen peptide, in particular such a preparation comprises at least 90 wt. %, preferably at least 95 wt. %, in particular at least 98 wt. %, in particular at least 99 wt. %, preferably 100 wt. % of the type II collagen or type II collagen peptide. In a preferred embodiment, only type II collagens or type II collagen peptides of a specific size, i.e. of a specific molecular weight, thus, of a single molecular species, are present in a homogeneous preparation. In a preferred embodiment of the present invention, the recombinant type II collagen or type II collagen peptide is present in isolated form. In a particularly preferred embodiment of the present invention, the recombinant type II collagen or the type II collagen peptide is present free of other proteins or peptides, in particular free of other substances, for example impurities, in particular free of non-protein material, free of salts and/or free of other proteins or peptides.

The term “gelatin” is understood in the context of the present invention in a customary manner in the art, in particular as defined for example in WO 01/34646.

In the context of the present invention, the term “recombinant DNA” refers to an artificially produced or manipulated DNA molecule produced in vitro by genetic methods. In a preferred embodiment, the recombinant DNA is composed of components from different organisms of origin.

In the context of the present invention, the term “expression cassette” is understood to mean a DNA segment which is responsible for transcribing the information encoded in this segment into an RNA, in particular into an mRNA, and which has at least one promoter and one protein-coding nucleotide sequence, as a rule at least one promoter, at least one protein-coding nucleotide sequence and optionally a terminator.

In the context of the present invention, a “nucleotide sequence” is understood to be the sequence of nucleotides of a nucleic acid, in particular of a nucleic acid strand, in particular of a DNA or RNA strand. A “nucleotide sequence” is therefore to be understood both as an informational unit and as the DNA or RNA strand physically manifesting this information.

In the context of the present invention, an “expression system” is understood to be a system in which targeted and controlled protein biosynthesis can take place. According to the invention, the term “expression system” comprises both cell-free expression systems, in which the components necessary for protein biosynthesis are not present within a cell, i.e. protein biosynthesis takes place outside a cell, and cell-based expression systems, in which protein biosynthesis takes place within a living cell. In the context of the present invention, a cell-free expression system is preferably a lysate or an extract from E. coli, insect cells, wheat germ, tobacco cells or mammalian cells, in particular CHO cells or reticulocytes from rabbits, which has the components necessary for protein biosynthesis, in particular a translation and a transcription system. In the case of using a cell-free expression system in one of the methods according to the present invention, the term “cultivating” is synonymous with “incubating”.

In the context of the present invention, the term “host cell” is understood to mean a living cell capable of expressing peptides or proteins encoded in foreign DNA, in particular in recombinant DNA.

According to the invention, the term “obtaining the type II collagen, in particular type II collagen peptide”, according to method step c), means a method known to the skilled person for isolating the type II collagen or type II collagen peptide from a composition containing several components by means of known isolation methods, such as, for example, centrifugation methods, in particular differential centrifugation and/or density gradient centrifugation, chromatographic methods, in particular gel filtration, ion exchange, affinity and/or high performance liquid chromatography, electrophoresis methods, filtration methods and/or extraction methods, wherein enrichment and purification of the component in question from the multi-component composition can preferably be achieved by sequential application of several isolation methods. Where appropriate, cleavage of C- and/or N-terminal procollagen fragments to obtain collagen may be carried out before, after or during the obtaining.

Preferably, within the conditions of method step b), fibrillogenesis, chemical modifications and secretion of the expressed type II collagen peptide may also occur.

According to the invention, “conditions enabling the expression of the type II collagen, in particular type II collagen peptide” are understood to mean conditions, such as in particular temperature, pressure, time, light and the presence or absence of inducers and/or repressors, which activate or enhance expression of the type II collagen, in particular type II collagen peptide. In a preferred embodiment, the expression of the type II collagen, in particular type II collagen peptide, takes place in the context of a high-cell-density fermentation, in particular under high pressure, preferably high air pressure. The specific conditions that enable expression of the type II collagen, in particular type II collagen peptide, are known to the person skilled in the art and depend on the expression system used and the expression cassette used, in particular the promoter contained therein. The expression of type II collagen, in particular type II collagen peptide, may be constitutive or inducible expression, depending on the structure of the expression cassette.

In the context of the present invention, a therapeutic method for oral therapy of cartilage diseases is understood to be a method for the prevention and/or treatment of cartilage diseases, in particular for the treatment of cartilage diseases, wherein the administration of the recombinant type II collagen is oral.

Cartilage diseases within the meaning of the present invention are in particular inflammatory, degenerative cartilage diseases and/or cartilage diseases caused by autoimmune actions, in particular by excessive immune reactions, in particular arthrosis and/or rheumatoid arthritis. In the context of the present invention, cartilage diseases are in particular joint cartilage diseases, especially of the joints in the feet, knees, fingers, wrists, hips and spine.

According to the invention, a therapeutic method for oral therapy of cartilage diseases is preferably also understood as a method for inducing oral tolerance to endogenous collagen, in particular endogenous type II collagen, in particular endogenous type II collagen present in or on cartilage tissue.

In the context of the present invention, the terms “comprising” and “having” are understood to mean that in addition to the elements explicitly covered by these terms, further elements not explicitly mentioned may be added. In the context of the present invention, it is also understood by these terms that only the explicitly mentioned elements are covered and that no further elements are present. In this particular embodiment, the meaning of the terms “comprising” and “having” is synonymous with the term “consisting of”. In addition, the terms “comprising” and “having” also cover compositions which, in addition to the explicitly mentioned elements, also include further elements which are not mentioned but which are of a functionally and qualitatively subordinate nature. In this embodiment, the terms “comprising” and “having” are synonymous with the term “consisting essentially of”.

Where, in the context of the present invention, the first and second decimal places or the second decimal place are/is not indicated, they/it are/is to be set as 0.

In the context of the present invention, the term “and/or” is understood to mean that all members of a group which are connected by the term “and/or” are disclosed both alternatively to each other and each among themselves cumulatively in any combination. This means for the expression “A, B and/or C” that the following disclosure is to be understood thereunder: a) A or B or C or b) (A and B) or c) (A and C) or d) (B and C) or e) (A and B and C).

Further preferred embodiments will be apparent from the subclaims.

The invention is described below, without limiting the general idea of the invention, by means of exemplary sequences, figures and embodiments.

### Designated Thereby:

SEQ ID No. 1: The coding nucleotide sequence of bovine type II collagen (CP90) (col2 α1; 3036 base pairs).
SEQ ID No. 2: The amino acid sequence of bovine type II collagen (CP90) (1012 amino acids).
SEQ ID No. 3: The coding nucleotide sequence of a collagen peptide derived from bovine type II collagen (col2 α1) (CP45) (1500 base pairs).
SEQ ID No. 4: The amino acid sequence of a collagen peptide derived from bovine type II collagen (CP45) (500 amino acids).
SEQ ID No. 5: The coding nucleotide sequence of a monomeric prolyl 4-hydroxylase (P4H) from mimi virus with N-terminal pOst1 signal sequence, 6×His-tag and C-terminal ER retention sequence HDEL.
SEQ ID No. 6: The amino acid sequence of a monomeric prolyl 4-hydroxylase (P4H) encoded by SEQ ID No. 5 from mimi virus (254 amino acids).
SEQ ID No. 7: The nucleotide sequence of plasmid pAOXsec-ColII-1.
SEQ ID No. 8: The nucleotide sequence of plasmid pAOXsec-ColII-1 s.a.
SEQ ID No. 9: The nucleotide sequence of plasmid pAOX_Mimi-int 3.0.

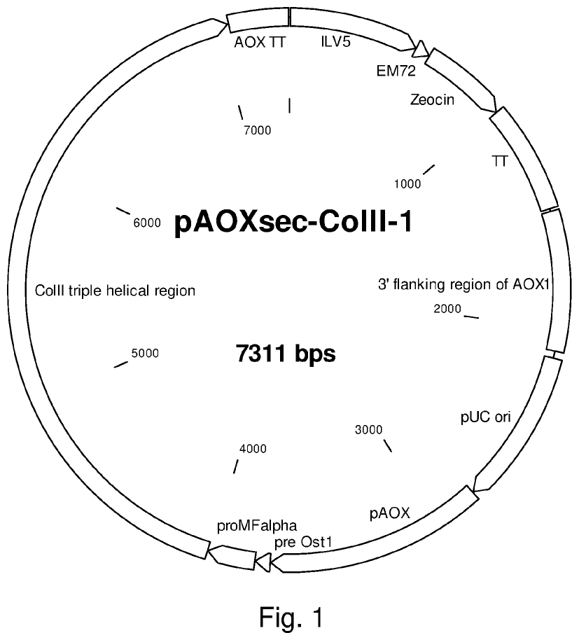

FIG. 1: A plasmid map of pAOXsec-ColII-1.

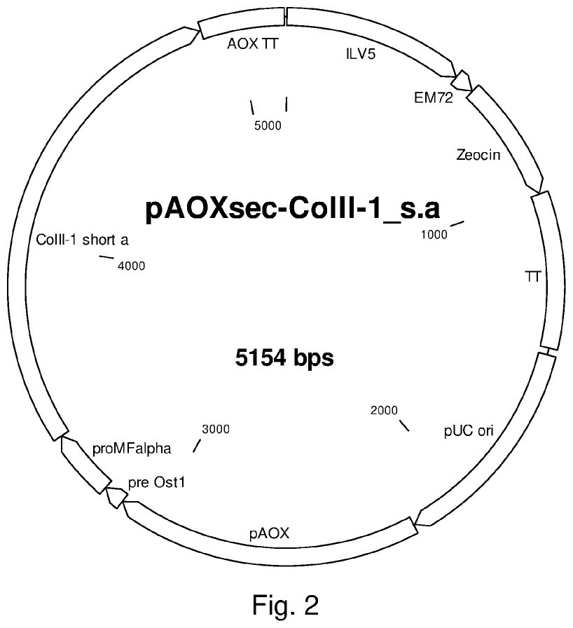

FIG. 2: A plasmid map of pAOXsec-ColII-1 s.a.

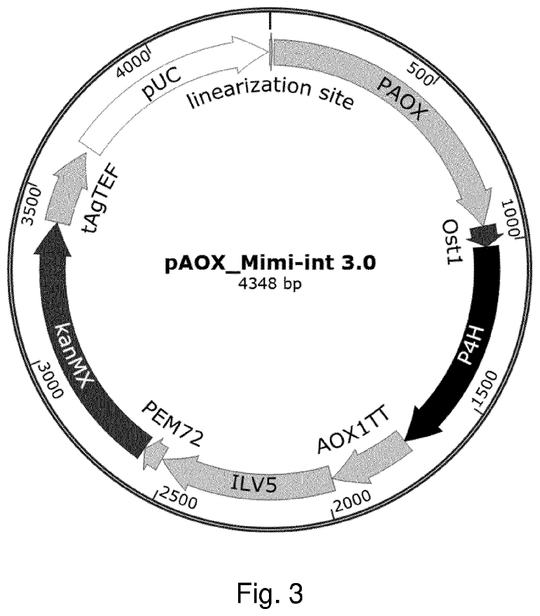

FIG. 3: A plasmid map of pAOX_Mimi-int 3.0.

### EXAMPLE 1

### Production of Recombinant Type II Collagen

Recombinantly produced hydroxylated full-length type II collagen with the amino acid sequence according to SEQ ID No. 2 (CP90) was obtained by recombinant expression of an expression cassette having the nucleotide sequence according to SEQ ID No. 1 in a Pichia pastoris strain capable of hydroxylating proline residues.

Furthermore, a recombinantly produced hydroxylated collagen peptide based on type II collagen having the amino acid sequence according to SEQ ID No. 4 (CP45) was obtained by recombinant expression of an expression cassette having the nucleotide sequence according to SEQ ID No. 3 in a Pichia pastoris strain capable of hydroxylating proline residues.

The Pichia pastoris strains used for the recombinant expression of CP90 and CP45, respectively, were obtained by genomic integration of the coding nucleotide sequence of bovine type II collagen (CP90) and the coding nucleotide sequence of a collagen peptide derived from bovine type II collagen (col2α1) (CP45) using the integration plasmids pAOXsec-ColII-1 (FIG. 1) and pAOXsec-ColII-1 s. a (FIG. 2) and the coding nucleotide sequence of a monomeric prolyl-4-hydroxylase from mimi virus (PH4) using the integration plasmid pAOX_Mimi-int 3.0 (FIG. 3).

### EXAMPLE 2

### Detection of Effectiveness

### A) Immune and Cytokine Modulation by Recombinant Type II Collagen

The immune-modulatory effect of native recombinantly produced type II collagen (recombinantly produced full-length type II collagen) according to Example 1 was determined in commercial healthy murine Peyer's patch M cells (SCC142M, Sigma Aldrich, Germany).

The M cells were cultivated in ITES-ERDF medium, which promotes the production of immunoglobulins. The culture medium was supplemented with 10% fetal calf serum, 10 μg/mL insulin, 20 μg/mL transferrin, 20 μM ethanolamine and 25 nM selenite (ITES). The synthesis of immunoglobulins in the cell culture supernatant was determined by specific enzyme-linked immunosorbent assays against IgE, IgA, IgM and IgG.

In addition, the effect of collagen peptides on the formation of pro-inflammatory (TNFα, IL-6, IFNγ) and anti-inflammatory cytokines (IL-4, IL-10, TGF-ß1) was investigated compared to untreated controls. RNA expression of pro-inflammatory and anti-inflammatory cytokines was tested after a cultivation period of 1-5 days.

B) Immunisation of DBA/1 Mice with Type II Collagen and Induction of Rheumatoid Arthritis.

10 mg recombinant type II collagen was diluted in 2.5 mL 0.01 N acetic acid solution to a final concentration of 4 mg/mL collagen (stock solution). The collagen was completely dissolved by rotating the reaction vessel overnight at 4° C.

The collagen stock solution was dissolved in 0.01 N acetic acid at a ratio of 1:1 (v/v) to produce a working solution of type II collagen. Complete Freund's adjuvant (CFA) was added to the collagen working solution at a ratio of 1:1 (v/v).

8-week-old male DBA/1J mice were kept at 20±2° C. under 12/12 h light-dark cycle and 55±10% humidity and standard laboratory rodent diet and water ad libitum.

Oral tolerance was induced by feeding 2 mg/mL of native recombinant type II collagen (100 μg dissolved in 0.05 N acetic acid) every second day as part of a two-week intervention and compared with an equal amount of phosphate-buffered saline (PBS) as a placebo control. Mice obtained six consecutive administrations of the collagen prior to subsequent induction of rheumatoid arthritis.

Mice were anaesthetised by intraperitoneal injection of 100 μL of ketamine-xylazine solution in phosphate-buffered saline (1:100 v/v; 62.5 mg ketamine, 0.625 mg xylazine in 10 mL PBS). Each mouse obtained 100 μL of the final CFA collagen solution subcutaneously into the tail, 2 cm anterior to the base of the tail, without penetrating blood vessels, to induce rheumatoid arthritis (RA).

Immunisation of the mice against type II collagen was boosted by repeating the injection after three weeks keeping. The boost injection was administered closer to the base of the tail to enhance the development of rheumatoid arthritis.

### C) Evaluation:

2.5 weeks after primary immunisation, the degree of arthritis was analysed three times a week for up to 12 weeks. The severity of arthritis was rated on a scale of 0 to 4:

0=no oedema or swelling; 1=mild oedema and erythema confined to the foot and/or ankle; 2=mild oedema and erythema from the ankle to the tarsal bone; 3=moderate oedema and erythema from the ankle to the tarsal bone; and 4=oedema and erythema from the ankle to the entire leg.

Blood samples taken from each mouse at 3, 5 and 7 weeks after the start of immunisation were used to test immunoglobulin concentrations using ELISA quantification kits. The formation of IgG, IgE, IgM and IgA was quantified photometrically by measuring the optical density at 415 nm.

At the end of the observation period, mice were sacrificed and Peyer's plaque M cells were isolated and cultivated in ITES-ERDF medium as described above.

RNA expression of cytokines and synthesis of immunoglobulins in murine M cells immunised with type II collagen and the PBS controls were determined as described above.

Cell culture experiments and immunotolerance tests in DBA/1J mice were repeated with triple helical fragments of native recombinant type II collagen and truncated non-helical fragments to analyse whether original helical collagen organisation is necessary to induce immunotolerance to endogenous type II collagen.

### D) Results:

The data determined showed an advantageous effect of recombinantly produced type II collagen in murine Peyer's plaque cells.

In healthy M cells, the RNA expression profile of cytokines showed an anti-inflammatory effect of recombinantly produced type II collagen. Furthermore, in healthy M cells, the synthesis of immunoglobulins was suppressed.

It was also shown that the synthesis of pro-inflammatory cytokines was suppressed and that of anti-inflammatory cytokines was also stimulated in immunised murine M cells, i.e. after immunisation against rheumatoid arthritis. Furthermore, the synthesis of immunoglobulins was suppressed in Peyer's plaque cells after immunisation against rheumatoid arthritis, as shown in Peyer's plaque isolated from murine intestinal tissue samples.

CFA-induced rheumatoid arthritis was reduced in vivo, as shown by a lower degree of arthritis in RA affected ankles in mice after immunisation with recombinant type II collagen compared to placebo control.

A reduced concentration of Ig's in blood samples from mice immunised with collagen confirmed these findings.

The data determined on truncated fragments of recombinant native type II collagen suggest that both triple-helical and non-helical fragments are sufficient to induce immune tolerance to rheumatoid arthritis inducing type II collagen.

### EXAMPLE 3

### Detection of Effectiveness

### A) Immune and Cytokine Modulation by Type II Collagen Peptides

The effects of the recombinantly produced type II collagen peptides CP90 and CP45 according to Example 1 with regard to an immune-modulatory effect were tested in further experimental approaches. Commercially obtainable human peripheral blood monocytes (PBMC), from the company 3H-Biomedical AB (Sweden), were used for the investigations.

Initially, PBMC cells were cultivated in macrophage base medium DXF (C-28057, PromoCell, Germany) in cell culture flasks coated with human fibronectin—(C-43060, PromoCell, Germany). The culture medium was supplemented with the appropriate supplement mix (supplement to C-28055, PromoCell, Germany) as well as 1% amphotericin and 1% penicillin-streptomycin. Redifferentiation of adherent monocytes to immunosuppressive macrophages of type 2b or 2c was induced by the addition of 4 μg/mL recombinant type II collagen. The cells were each incubated with the recombinant collagen peptides for 6 days. The activated macrophages were then polarised by the addition of 1 μg/mL lipopolysaccharides from Escherichia coli (LPS, L6529, Merck, Germany). Subsequently, the differentiation pattern of the generated macrophages was analysed using specific cell differentiation markers (CDs). The differentiation of monocytes into inflammation-inducing M1 macrophages or into immunosuppressive M2 macrophages was detected using specific markers. For this purpose, the M2 surface markers CD86, CD14 and CD163 were determined by ELISA (“Enzyme-Linked Immunosorbent Assay”). The respective detections for CD86 (850590096 Diaclone, Hölzel Diagnostics, Germany), CD14 (850780096 Diaclone, Hölzel Diagnostics, Germany) and CD163 (ELH-CD163 RayBiotech, Hölzel Diagnostics) were performed exactly according to the manufacturer's instructions. To exclude differentiation to inflammatory M1 macrophages, the M1 macrophage markers CD86 (850590096 Diaclone, Hölzel Diagnostics, Germany) and CD80 (EK0707 Boster PicoKine, Hölzel Diagnostics, Germany) were additionally analysed.

Furthermore, the effect of the type II collagen peptides on the formation of pro-inflammatory (TNFα, IFNY) and anti-inflammatory cytokines (IL-10) in the culture supernatant was analysed. TNF (EK0525 Boster PicoKine, Hölzel Diagnostics, Germany), IL-10 (950060096, Diaclone, Hölzel Diagnostics, Germany) and IFNY (EK0373, Boster PicoKine, Hölzel Diagnostics, Germany) were determined by ELISA technique according to manufacturer's instructions.

Differentiation of monocytes to M1 or M2 macrophages was checked using a special differentiation medium (C-28055, PromoCell, Germany) and specific cultivation additives.

The type II collagen peptides CP90 and CP45 produced according to example 1 with an average molecular weight of 90 and 45 kDa, respectively, were used for the studies.

The data obtained showed a statistically significant (p<0.05), advantageous effect of the type II collagen peptides CP90 and CP45 used on the differentiation into immunosuppressive M2 macrophages from peripheral blood monocytes.

In differentiated macrophage cells, an overall anti-inflammatory effect of the tested type II collagen peptides could be demonstrated on the basis of the synthesis profile of specific cytokines. Thus, the synthesis of inflammatory cytokines was statistically significantly (p<0.05) reduced and the synthesis of anti-inflammatory IL-10 was statistically significantly (p<0.05) induced.

### EXAMPLE 4

### Stimulation of Naive CD4⁺ T Precursor Cells

After induction of monocytes into M2 macrophages by CP90 or CP45 (Example 3), the macrophage base medium DXF (C-28057, PromoCell, Germany) was exchanged for T cell culture medium (3H800-50-50, 3H Biomedical AB, Sweden). Naive CD4⁺ T precursor cells (3H31-k, 3H Biomedical AB, Sweden) were added to the differentiated M2 macrophages. Through direct cell-cell contacts of naive T precursor cells with the differentiated M2 macrophages and their cytokine cocktail, the T precursor cells differentiate into regulatory T suppressor cells.

To significantly increase the number of specific T cell clones, the mature T suppressor cells were enriched using the ARTE (antigen-reactive T-cell enrichment) method. In this method, the specification of the T cells is determined by labelling the cells with cell surface marker (CD) antibodies coupled to various dyes such as biotin or phycoerythrin. To enrich the specific T cell clone types, they were then separated using anti-biotin and anti-PE coupled magnetic MicroBeads.

Afterwards, the T cells could be stained with fluorochrome-conjugated antibodies and quantified by flow cytometry. T suppressor cells are identified by forkhead box p3 (FoxP3) and CD25.

It was shown that supplementation of recombinant type II collagen peptide statistically significantly (p<0.05) increased the release of anti-inflammatory cytokines such as IL-10, IL-4 and TGF-ß by differentiated T-suppressor cells.

The data obtained also showed a significantly advantageous effect of the recombinantly produced type II collagen peptides CP90 and CP45 on the formation of immunosuppressive T-suppressor cells.

### EXAMPLE 5

### Immunosuppression in Articular Chondrocytes

Human articular chondrocytes were cultivated in Hams-F12 medium (HAM-12-A, Capricorn, Germany) supplemented with 1% amphotericin and 1% penicillin-streptomycin and 10% calf serum. After reaching 100% cell confluence, an inflammatory situation was induced in the chondrocytes by adding 1 μg/ml lipopolysaccharide (Escherichia coli, L6529, Merck, Germany). By adding 25 μl/ml cell supernatant of the T-cell differentiation experiment (see example 4), the inflammation in the chondrocytes was reduced.

Based on the statistically significantly reduced expression of pro-inflammatory cytokines (IL-1ß, TNFα and IL-6) in the chondrocytes, the anti-inflammatory effect of the investigated type II collagen peptide CP90 could be clearly demonstrated by real-time PCR.

In another experiment with an identical experimental set-up, the anti-inflammatory effect of a shorter, recombinantly produced type II collagen peptide (CP45) was tested. Here, as in the investigations of CP90, a statistically significant (p<0.05) reduction in the expression of pro-inflammatory cytokines (IL-1ß, TNFα and IL-6) in the chondrocytes could also be demonstrated for CP45. Thus, a corresponding anti-inflammatory effect could also be shown for the type II collagen-based CP45.

A comparison between the type II collagen peptides CP90 and CP45 showed no statistically significant difference with regard to the anti-inflammatory effect of both collagen peptides analysed.

In summary, the data determined show an advantageous effect of the recombinantly produced type II collagen peptides CP90 and CP45 through the statistically significant (p<0.05) increased production of anti-inflammatory cytokines in immunosuppressor T cells. Furthermore, the results support the underlying mode of action in terms of oral tolerance induction by oral application of type II collagen, or type II collagen peptides (CP90 and CP45).

1. A method for oral therapy of a cartilage disease of a human or animal patient, comprising administering to the patient a recombinant type II collagen.

2. The method according to claim 1, wherein the recombinant type II collagen is bovine type II collagen, preferably bovine type II collagen comprising the amino acid sequence according to SEQ ID No. 2 or according to SEQ ID No. 4.

3. The method according to claim 1, wherein the recombinant type II collagen is present in the form of type II procollagen or mature type II collagen.

4. The method according to claim 1, wherein the recombinant type II collagen is present in triple-helical form, in particular is present in the form of a homotrimer of type II-al chains.

5. The method according to claim 1, wherein the recombinant type II collagen is present in the form of cross-linked or non-cross-linked fibrils.

6. The method according to claim 1, wherein the recombinant type II collagen is present in the form of a type II collagen peptide.

7. The method according to claim 6, wherein the recombinant type II collagen peptide has a molecular weight in a range of 35 to 95 kDa.

8. The method according to claim 1, wherein the recombinant type II collagen is non-denatured.

9. The method according to claim 1, wherein the recombinant type II collagen is present fully or partially hydroxylated, fully or partially glycosylated, or fully or partially hydroxylated and glycosylated.

10. The method according to claim 1, wherein the recombinant type II collagen has been produced by expression in a eukaryotic host cell, in particular a yeast cell, preferably Pichia pastoris, or prokaryotic host cell, in particular E. coli, in particular in hydroxylated form and/or in the form of a fusion peptide.

11. The method according to claim 1, which is present as a recombinant type II collagen peptide in isolated, homogeneous form with uniform molecular weight, in a mixture of type II collagen peptides or in a hydrolysate of a recombinant type II collagen, in particular recombinant type II collagen peptide.

12. The method according to claim 1, wherein the type II collagen is the type II collagen of a vertebrate, in particular pig, sheep, cattle, rodent, kangaroo, horse, bird, reptile, amphibian, or fish, or of an invertebrate, in particular jellyfish.

13. The method according to claim 1, wherein the cartilage disease is an immune-modulated cartilage disease, in particular an autoimmune disease, in particular polychondritis or rheumatoid arthritis.

14. The method according to claim 1, wherein the cartilage disease is an inflammatory or degenerative cartilage disease, in particular arthrosis and/or rheumatoid arthritis.

15. The method of claim 1, wherein the recombinant type II collagen is provided in a composition further comprising at least one pharmaceutically acceptable or food-acceptable carrier and, optionally, at least one additive or excipient.

16. The method according to claim 15, wherein the at least one excipient is chondroitin, chondroitin sulphate, hyaluronic acid, aflapin, univestin, 5-loxin, glucosamine, glucosamine sulphate or methylsulphonylmethane (MSM).

17. The method according to claim 15, wherein the at least one additive is a recombinantly produced collagen hydrolysate, a collagen hydrolysate originated from natural sources, a recombinantly produced collagen I, a collagen I obtained from natural sources, or a combination thereof.

18. The method according to claim 15, wherein the composition is present in the form of a tablet, lozenge, chewable tablet, powder, granules, hard capsule, soft capsule, capsule, bite capsule, dragee, pastille, extrudates, juice, suspension or gel.

19. The method according to claim 15, wherein the composition is suitable for oral administration at a dose of 1 to 60 mg/day of recombinant type II collagen.

20. A method for the therapeutic treatment or therapeutic prophylaxis of immune intolerance to type II collagen, in particular immune-modulated cartilage disease, or for obtaining cartilage health in a subject, comprising administering the subject a recombinant type II collagen, in particular a recombinant type II collagen peptide.

21. (canceled)
